(19) United States
(12) Patent Application Publication
(10) Pub. No.: US 2023/0262436 A1
(43) Pub. Date: Aug. 17, 2023

(54) PERSONAL DEVICE, A SYSTEM AND METHODS FOR TRANSMITTING DATA ASSOCIATED WITH THE PERSONAL DEVICE

(71) Applicant: Pink Nectarine Health AB, Stockholm (DE)

(72) Inventors: Tobias LINGHAMMAR, Enebyberg (SE); Martin LINREY, Sollentuna (SE)

(73) Assignee: Pink Nectarine Health AB, Stockholm (SE)

(21) Appl. No.: 18/015,283

(22) PCT Filed: Jul. 9, 2021
(86) PCT No.: PCT/SE2021/050713

§ 371 Date: Jan. 9, 2023

(30) Foreign Application Priority Data
Jul. 9, 2020 (SE) 2050879-2

(51) Int. Cl. H04W 4/80; H04W 76/14; A61B 5/00

(52) U.S. Cl. CPC H04W 4/80; H04W 76/14; A61B 5/746; A61B 5/6801; A61B 5/002; H04W 84/18

(57) ABSTRACT

Methods, a system, a self-organizing network of nodes and a plurality of portable personal devices are disclosed. The personal devices are adapted to be worn by individuals and are configured to transmit short-range wireless broadcast signals while a short-range wireless connection between the personal device and a node of the network of nodes is active. The controller node assigned to a personal device may then be configured to determine the position or location of the individual and take actions on behalf of the individual wearing the personal device in response to received data contained in broadcast signals forwarded by other nodes and/or status data, sensor data and/or event data received and forwarded to the controller node by the connection node assigned to the personal device.

### TECHNICAL FIELD

The present invention relates to methods, a system, a self-organizing network of nodes and a portable personal device. Specifically, the present invention relates to methods for transmitting data associated with a personal device worn by an individual both via an established short-range wireless connection link between the personal device and a network node and via short-range wireless broadcasts from the personal device. and

### BACKGROUND

Systems for monitoring such individuals, e.g. elderly, sick or injured people, and automatically issuing alarms have been used for some time. Monitoring systems for monitoring such individuals often comprise a personal device (e.g. a wristband) comprising sensors, the signals of which are analysed to determine whether help is needed, e.g. due to a fall.

U.S. Pat. No. 6,433,690 discloses a method and system for recording acceleration and body position data from elderly or disabled persons. The fall monitoring system includes signal feature extraction and interpretive methods for characterizing accelerations and body positions during fall events. The system can detect health and life-threatening fall events in elderly persons and can autonomously notify nursing personnel or family members that the person is in need of immediate assistance. The monitoring of a person's fall is performed by using an accelerometer in a monitoring device carried on the person, which monitoring device samples the person's body angle and body acceleration.

U.S. Pat. No. 9,668,297 describes a BLE scatternet system and method that uses a schedule for when advertisements should be transmitted. The advertiser transmits an initial advertisement, receives a connection request, and then goes to sleep to wake up and transmit subsequent advertisements according to the schedule.

U.S. Ser. No. 10/313,866 describes a method for establishing and monitoring a Bluetooth low energy wireless network by using the broadcast state.

US2019/0215244 describes a Self-Organizing Network (SON) for Internet of Things (I), where an IoT device may be selected to be an “IoT coordinator”. A coordinator may e.g. perform the roles of managing a neighboring IoT device and forwarding a packet. This SON method includes receiving neighboring device information from at least one neighboring IoT device of an IoT device, updating device information of the IoT device based on the received neighboring device information, and determining whether the IoT device is a coordinator candidate based on at least one of a number of network interfaces, a number of neighbor links connected to the network interfaces, or a ratio of remaining energy, which is included in the device information.

The managing of the IoT devices in US2019/0215244 is performed by neighboring IoT devices and, thus, relies on the presence of other IoT devices within short-range communication range and is also sharing short-range wireless transmission medium and protocol with other IoT devices thus is dependent on the currently available short-range bandwidth of those neighboring IoT devices, e.g. the current number of other links connected to the network interface of those neighboring IoT devices.

There is a need for a more scalable and load-balanced system and network which enables both faster responses to events and sensor data and provides a higher bandwidth capacity for the communication between the personal devices and the units making the decisions on behalf of the personal devices, e.g. the control units used for collecting data for monitoring the individuals.

More specifically, there is a need for a more scalable system comprising a self-organizing network which does not rely on the presence of other IoT devices within short-range communication range for making decisions on behalf of other IoT devices and which is less dependent on sharing short-range wireless transmission medium, interface and protocol with other IoT devices. As an example, there is a need for a system and network which is less dependent on the currently available short-range bandwidth of those neighboring IoT devices, e.g. the current number of other links connected to the network interface of those neighboring IoT devices.

### Problems with the Prior Art

It is difficult to design a system for monitoring a group of personal devices carried by individuals moving between different locations or rooms of a facility which is highly scalable and provides reliable and fast responses to obtained sensor data, event data and inputs received from the monitored individuals as well as accurate and reliable positioning of the individuals, yet is robust in that each of the personal devices always has a reliable short-range wireless connection to the node network which compete less for bandwidth.

There is thus a need for an improved system and method for monitoring a plurality of individuals within a facility.

### SUMMARY

The above described problem is addressed by the claimed system for monitoring individuals within predetermined facilities by transmitting data from personal devices worn by the individuals both via established short-range wireless connection links with the node network and via short-range wireless broadcasts.

The technology disclosed relates to methods, a system comprising a self-organizing network of nodes and a plurality of personal devices each configured to transmit data associated with the respective personal device both via established short-range wireless connection links with the node network and via short-range wireless broadcasts.

The technology disclosed also relates to a system and methods for transmitting data associated with a personal device worn by an individual both via an established short-range wireless connection link between the personal device and a network node and via short-range wireless broadcasts from the personal device.

In aspects, the personal device is adapted to be worn by an individual and is configured to transmit short-range wireless broadcast signals while a short-range wireless connection between the personal device and a node of the network of nodes is active.

In aspects, technology disclosed relates to methods and a system comprising at least two network nodes and a plurality of personal devices each configured to transmit at least one short-range wireless broadcast signal which is received by any of the at least two network nodes within short-range wireless communication range with the respective personal device. The transmission of the at least one short-range wireless broadcast signal is performed while an established communication connection or link between the personal device and one of the network nodes which is the assigned connection node for the respective personal device is active. The transmission of broadcast signals may be performed in parallel with or while the personal device is transmitting messages via the established connection or link.

In aspects, technology disclosed relates to methods and a system comprising at least three network nodes and a plurality of personal devices each configured to transmit at least one short-range wireless broadcast signal which is received by any of the at least three network nodes within short-range wireless communication range with the respective personal device. The transmission of the at least one short-range wireless broadcast signal is performed while an established communication connection or link between the personal device and one of the network nodes which is the assigned connection node for the respective personal device is active. The transmission of broadcast signals may be performed in parallel with or while the personal device is transmitting messages via the established connection or link.

In embodiments, the personal devices are adapted to be worn by individuals and are configured to transmit short-range wireless broadcast signals while a short-range wireless connection between the personal device and a node of the network of nodes is active. The controller node assigned to a personal device may then be configured to determine the position or location of the individual and take actions on behalf of the individual wearing the personal device in response to received data contained in broadcast signals forwarded by other nodes and/or status data, sensor data and/or event data received and forwarded to the controller node by the connection node assigned to the personal device.

According to aspects and certain embodiments of the technology disclosed, data or information contained in the at least one short-range wireless broadcast message received by the connection node via the established communication connection or link is forwarded via an IP-based network to the assigned controller node for the personal device The controller node, which has sole responsibility for the personal device, may be further configured to use data forwarded from the assigned connection node and/or data associated with the personal device which is received from the other nodes to take actions or make decisions on behalf of the personal device.

In aspects, the technology disclosed relates to a system including a network comprising at least two network nodes and a plurality of personal devices with short-range wireless communication capabilities. The system is configured to assign a connection node among the at least two network nodes to each of the plurality of personal devices. The connection node is responsible for establishing a short-range wireless communication link with the personal device it is assigned connection node for.

In aspects, the technology disclosed relates to a system including a network comprising at least three network nodes and a plurality of personal devices with short-range wireless communication capabilities. The system is configured to assign a connection node among the at least three network nodes to each of the plurality of personal devices. The connection node is responsible for establishing a short-range wireless communication link with the personal device it is assigned connection node for.

According to the technology disclosed, each of the plurality of personal devices is configured to transmit short-range wireless broadcast signals while their respective established short-range wireless connection with their respectively assigned connection node is active.

In embodiments, the system is configured to assign a controller node for the respective of the plurality of personal device, and wherein each controller node for the respective personal device is configured to assign a connection node for the personal device and to receive, from the other network nodes, status data, sensor data and/or event data associated with the personal device it is responsible for.

In embodiments, the network nodes are configured to share data with the other nodes via an IP-based network connecting at least two network nodes.

In embodiments, the network nodes are configured to share data with the other nodes via an IP-based network connecting at least three network nodes.

In embodiments, the network nodes are configured to share data with the other nodes via an IP-based network connecting at least five network nodes.

In embodiments, the IP-based network is a separate network and is configured to use a different communication protocol from the communication protocol used for the established short-range wireless communication link between the connection node and the personal device.

In embodiments, the established short-range wireless communication link between the connection node and the personal device is a bidirectional short-range wireless communication link.

In embodiments, each of the plurality of personal devices is configured to transmit data indicating the same obtained sensor data and/or event data both via their respective established short-range wireless connection with their respectively assigned connection node and via short-range wireless broadcast signals. The same sensor data and/or event data may then be data indicating at least one of the activation of an input means of the personal device, data indicating a trigger event and sensor data indicating a health state and/or alert state for the personal device and/or the individual wearing the personal device.

In embodiments, the controller node for the respective personal device is configured to determine a specific alert state and/or a health state for the individual wearing the personal device at least partly based on received sensor data and/or event data associated with the personal device.

In certain embodiments, the controller node for the respective personal device is configured to set or change a health state for the individual wearing a personal device at least partly based on received sensor data obtained by and/or originating from at least one medical sensor of the personal device.

In embodiments, the controller node currently assigned to a personal device is configured to transmit alert state data and/or alarm data to a local or remote processing arrangement, e.g. backend system, based on received sensor data and/or event data. The local or remote processing arrangement may then be configured to set an alarm or determine whether to set an alarm based on the received alert state data and/or alarm data by informing and/or transmitting an alarm to the staff at the facility, e.g. send an alarm message to the mobile communication devices belonging to the staff members at the facility.

In embodiments, the controller node currently assigned to a personal device is configured to transmit alert state data and/or alarm data to a local or remote processing arrangement, e.g. backend system, based on received sensor data and/or event data. The local or remote processing arrangement may then be configured to set an alarm or determine whether to set an alarm based on the received alert state data and/or alarm data by informing and/or transmitting an alarm to the staff at the facility, e.g. send an alarm message to the mobile communication devices belonging to the staff members at the facility.

In embodiments, each of the network nodes of the node network is configured to first measure signal strengths of short-range wireless broadcast signals received from a personal device and then automatically transmit or forward data indicating the measured signal strengths to the identified controller node responsible for the personal device transmitting the short-range wireless broadcast signals.

In embodiments, the controller node currently assigned to a personal device is configured to determine a position and/or a location for their respective personal device at least partly based on the received data indicating the measured signal strengths.

In embodiments, the controller node for the respective personal device is configured to set an alert state or alarm data based on received sensor data and/or event data and also determine a position and/or a location for their respective personal device at least partly based on the received data indicating the measured signal strengths.

In embodiments, the controller node for the respective personal device is configured to transmit alert state data and/or alarm data to a local or remote processing arrangement, e.g. backend system, based on received sensor data and/or event data and also determine a position and/or a location for their respective personal device at least partly based on the received data indicating the signal strengths of short-range wireless broadcast signals measured by the network nodes within short-range wireless communication range with the personal device transmitting the broadcast signals. The local or remote processing arrangement may then set an alarm or determine whether to set an alarm based on the received alert state data and/or alarm data by informing and/or transmitting an alarm to the staff at the facility where the alarm includes the determined position and/or location, e.g. send an alarm message to the mobile communication devices belonging to the staff members at the facility.

In embodiments, the controller node for the respective personal device is configured to set or change a health state for the individual wearing a personal device at least partly based on received sensor data obtained by and/or originating from at least one medical sensor of the personal device and also determine a position and/or a location for their respective personal device at least partly based on the received data indicating the measured signal strengths.

In embodiments, the determining of a position and/or location for the individual wearing the personal device by the controller node may be based on the received signal strength indication (RSSI) of short-range wireless broadcast signals transmitted by the personal device and which are received by the network nodes within short-range communication range with the personal device. The network nodes within short-range communication range then measure the RSSI of short-range wireless broadcast signals and forwards the RSSI to the controller node for the personal device. If the RRSI of short-range wireless broadcast signals received by more than one network node is determined, the location of the personal device may be more reliably determined based on e.g. triangulation. Machine learning may also be used for this determination. The measured RSSI values of short-range wireless broadcast signals transmitted by a personal device may be used by the controller node assigned to a personal device for determining the current position and/or location for the personal device in connection with transmitting position or location data together with alert state data or when setting an alarm for the individual wearing the personal device. Thus, the decision by the controller node for a particular personal device to transmit alert data or set an alarm for the individual may be based on broadcast signal strength values measured and received from other nodes within short-range communication range with the personal device (including the current controller node and the current connection node for the personal device if any of these two nodes are within short-range wireless communication range with the personal device to receive the broadcast signals).

In aspects, the technology disclosed relates to a method in a system including a network comprising network nodes and a personal device with short-range wireless communication capabilities, said method comprising:
a. assigning a network node to be controller node for the personal device;
b. assigning, by said controller node, a connection node for establishing a short-range wireless communication link between said connection node and said personal device;
c. establishing, by said assigned connection node, a short-range wireless communication link with said personal device;
d. transmitting, from said personal device and via said established short-range wireless communication link, a message to said connection node;
e. receiving, by said connection node, said message, wherein said message contains data associated with said personal device;
f. forwarding, from the assigned connection node to the controller node for said personal device, said message or data to said assigned controller node for said personal device; and
g. transmitting, from said personal device, a short-range wireless broadcast signal, wherein said short-range wireless broadcast signal is transmitted while said short-range wireless communication link between said connection node and said personal device is active, and wherein said short-range wireless broadcast signal is received or detected by any network node currently within short-range communication range with said personal device.

In aspects, the technology disclosed relates to a method in a system including a network comprising at least two network nodes and a plurality of personal devices with short-range wireless communication capabilities, the method comprising:
a. assigning, e.g. automatically by the at least two network nodes and based on a common decision model which is known to the at least two network nodes, one node among the at least two network nodes to be the controller node for a personal device;
b. assigning a connection node for establishing a short-range wireless communication link between said connection node and said personal device;
c. establishing, by the assigned connection node, a short-range wireless communication link with said personal device;
d. transmitting, from the personal device to the connection node via the established short-range wireless communication link, a message containing at least one of status data, sensor data and event data associated with the personal device;
e. receiving, by the connection node assigned to the personal device, said message;
f. transmitting, from the assigned connection node to the controller node for the personal device, data including said at least one of status data, sensor data and event data associated with the personal device; and
g. transmitting, from said personal device, at least one short-range wireless broadcast signal, wherein said at least one short-range wireless broadcast signal is transmitted while said short-range wireless communication link between said connection node and said personal device is active.

In aspects, the technology disclosed relates to a method in a system including a network comprising network nodes and a personal device with short-range wireless communication capabilities, said method comprising:
a. assigning a network node to be controller node for the personal device;
b. assigning, by said controller node, a connection node for establishing a short-range wireless communication link between said connection node and said personal device;
c. establishing, by said assigned connection node, a short-range wireless communication link with said personal device;
d. transmitting, from said personal device and via said established short-range wireless communication link, a message to said connection node;
e. receiving, by said connection node, said message, wherein said message contains data associated with said personal device;
f. forwarding, from the assigned connection node to the controller node for said personal device, said message or data to said assigned controller node for said personal device; and
g. transmitting, from said personal device, a short-range wireless broadcast signal, wherein said short-range wireless broadcast signal contains data associated with said personal device and is transmitted while said short-range wireless communication link between said connection node and said personal device is active, and wherein said short-range wireless broadcast signal is received or detected by any network node currently within short-range communication range with said personal device; and
h. forwarding, by any and all network nodes currently within short-range communication range with said personal device and to said controller node for said personal device, at least one of data extracted from said short-range wireless broadcast signal and measured, by each of the network nodes received the broadcast signal, respective signal strengths of the received short-range wireless broadcast signal.

In embodiments, said at least one of status data, sensor data and event data associated with said personal device is provided, e.g. automatically provided, to said controller node by forwarding said received message from said connection node or by transmitting, from said connection node, a message including data corresponding to, or at least partly based on, said received at least one of status data, sensor data and event data associated with said personal device.

In embodiments, said message is forwarded or transmitted via an IP-based network connecting the network nodes.

In embodiments, said IP-based network is a separate network and uses a different communication protocol from the communication protocol used for said established short-range wireless communication link between said connection node and said personal device.

In embodiments, said established short-range wireless communication link between said connection node and said personal device is a bidirectional short-range wireless communication link.

In embodiments, said message from said connection node to said controller node and said at least one short-range wireless broadcast signal transmitted from said personal device are containing data indicating the same sensor data and/or event data.

In embodiments, the message from the connection node to the controller node is containing data indicating at least one of the activation of an input means of the personal device, data indicating a trigger event and sensor data indicating a health state and/or alert state for the personal device and/or the individual wearing the personal device.

In embodiments, the at least one short-range wireless broadcast signal transmitted from the personal device is containing data indicating at least one of the activation of an input means of the personal device, data indicating a trigger event and sensor data indicating a health state and/or alert state for the personal device and/or the individual wearing the personal device.

In embodiments, the message from transmitted from the connection node is containing sensor data and/or event data, and the method is further comprising:
a. determining, by the controller node and at least partly based on received sensor data and event data associated with the personal device, a specific alert state and/or a health state for the individual wearing the personal device.

In embodiments, the message transmitted from the connection node is containing sensor data and/or event data, and the method is further comprising:
b. transmitting, from the controller node to a local or remote processing arrangement, at least one of said sensor data and/or event data and data indicating a changed health state and/or alert state for the individual wearing the personal device.

In embodiments, the message transmitted from the connection node is containing sensor data and/or event data, and the method is further comprising:
c. transmitting, from the controller node, the sensor data and/or event data associated with the personal device to a local or remote processing arrangement; and
d. determining, by the local or remote processing arrangement, the current health state and/or alert state for the individual wearing the personal device based on said sensor data and/or event data, wherein processing of the sensor data for determining the current health state and/or alert state is performed by the local or remote processing arrangement using a trained machine learning algorithm.

In embodiments, the method is further comprising:
e. determining, by the local or remote processing arrangement, a changed health state and/or alert state for the individual wearing the personal device controller node, wherein the determining is at least partly based on said received sensor data and event data associated with the personal device is indicating a specific alert state and/or a health state for the individual wearing the personal device; and
f. transmitting, from the controller node to a local or remote processing arrangement, data indicating the determined changed health state and/or alert state for the individual wearing the personal device.

In embodiments, the method is further comprising:
g. setting, by the local or remote processing arrangement, an alarm for the personal device and/or individual wearing the personal device, wherein said setting of an alarm is at least partly based on the determining of a changed health state and/or alert state for the individual wearing the personal device.

In embodiments, the method according to the technology disclosed is comprising:
a. receiving, by at least one network node within short-range wireless communication range with the personal device, at least one short-range wireless broadcast signal;
b. measuring, by each of the at least one network node within short-range wireless communication range with the personal device, the signal strengths of the at least one short-range wireless broadcast signal;
c. transmitting, from each of the at least one network node within short-range wireless communication range with the personal device and to the controller node, at least one message containing data indicating the respective signal strengths of the at least one short-range wireless broadcast signal measured by the respective at least one network node within short-range wireless communication range with the personal device; and
d. receiving, by the controller node for the personal device, at least one message containing data indicating the respective signal strengths, wherein a message is received from the respective of the at least one network node and is indicating the respective signal strengths measured by the respective at least one node within short-range wireless communication range with the personal device.

In embodiments, the method is further comprising:
h. determining, by the controller node and at least partly based on the data indicating measured signal strengths, the current position and/or location for the personal device and/or the individual wearing the personal device.

In embodiments, the determining of the current position and/or location for the personal device and/or the individual wearing the personal device is in addition based on sensor data obtained by a motion sensor and/or pressure sensor of the personal device.

In embodiments, the method is further comprising:
i. transmitting, from the controller node to a local or remote processing arrangement, data indicating the measured signal strengths for the respective at least one node; and
j. determining, by the local or remote processing arrangement, the current position and/or location for the personal device and/or the individual wearing the personal device, wherein said determining is at least partly based on received data indicating measured signal strengths for the respective at least one node.

In embodiments, the method is further comprising:
k. obtaining, by at least one of a motion sensor and a pressure sensor of the personal device, sensor data indicating a pressure and/or a movement associated with the personal device and/or the individual wearing the personal device; and
l. transmitting, from the personal device to the connection node via the established short-range wireless communication link, data indicating said sensor data obtained by the personal device;
m. receiving, by the connection node, data indicating said sensor data obtained by the personal device;
n. forwarding, by the connection node to the controller node, data indicating said sensor data obtained by the personal device; and
o. determining, by the controller node, the current position and/or location for the individual earing the personal device, wherein the determining of the current position and/or location is at least partly based on said sensor data obtained by a motion sensor and/or a pressure sensor of the personal device.

In embodiments, the message from the connection node is containing sensor data and/or event data, and the method is further comprising:
p. transmitting, from the controller node, said sensor data and/or event data associated with the personal device to a local or remote processing arrangement; and
q. determining, by the local or remote processing arrangement, the current health state and/or alert state for the individual wearing the personal device based on said sensor data and/or event data, wherein the processing of the sensor data for determining the current health state and/or alert state is performed by the local or remote processing arrangement using a trained machine learning algorithm.

In embodiments, the method is further comprising:
r. determining, by the controller node, a changed health state and/or alert state for the individual wearing the personal device controller node at least partly based on received sensor data and event data associated with the personal device is indicating a specific alert state and/or a health state for the individual wearing the personal device; and
s. transmitting, from the controller node to a local or remote processing arrangement, data indicating a changed health state and/or alert state for the individual wearing the personal device.

The technology disclosed provides a solution for acting on an identified event by transmitting (redundant) event data, e.g. event/alarm data which is triggered by pressing a button on a wristband or triggered by voice activation of a voice activation means of the personal device, both via an established short-range wireless connection with one of the nodes and via at least one short-range wireless broadcast signal or message, e.g. Bluetooth broadcast signals or messages, from the personal device and where the short-range wireless broadcast signal are received by the network nodes within short-range wireless communication range with the personal device.

In embodiments, the technology disclosed provides a solution for fast response to obtained sensor data or an identified event, e.g. health sensor data indicating a health state, e.g. a deteriorated health condition, of the individual wearing the personal device or the event of pressing an alarm button on a personal device such as a wristband. The fast response solution according to the technology disclosed is achieved by the transmission of sensor data and/or event data indicating health sensor data, e.g. obtained by a medical sensor, or the occurrence of an event such as the pressing an alarm button on the personal device or activating an voice activation means, both via an established short-range wireless connection with one of the network nodes and via short-range wireless broadcast transmissions, e.g. via Bluetooth, from the personal device and which are received by those network nodes which are within short-range wireless communication range, e.g. Bluetooth range, with the respective personal device transmitting the broadcast signals.

In embodiments, the technology disclosed provides a solution for dynamically and accurately determining the indoor position or location for a personal device by measuring, by each of the network nodes which are within short-range communication range with the personal device, the signal strength of short-range wireless broadcast signals, e.g. Bluetooth broadcast signals, received from the personal device.

In embodiments, the controller node for the personal device may then receive, via the IP-based network, data and/or information representing or reflecting the measured signal strengths from each of the plurality of nodes within short-range communication range with the personal device and, based on the data and/or information (including signal strengths measured by the current controller node itself and/or the current connection node for the personal device if any of these nodes are within short-range wireless communication range with the personal device to receive the broadcasts), determine the current position or location for the personal device.

In certain embodiments, the controller may in addition use other obtained sensor data, e.g. sensor data obtained from the movement sensor of the personal device, e.g. an accelerometer, to determine and/or calculate the current position and/or location for the personal device.

In certain embodiments, the controller node may forward the obtained data and/or information representing or reflecting the measured signal strengths received from the network nodes within short-range communication range with the personal device, and/or other movement sensor data obtained, to a locally-installed or remote processing arrangement such as a backend system and network which determines the current position or location for the personal device.

In certain embodiments, the current position or location, e.g. room, for the personal device may at least partly be determined by a trained machine learning algorithm used by the controller node or a locally-installed or remote processing arrangement such as a backend system communicatively coupled to the controller node, since machine learning is an efficient way of evaluating complex signals in situations where large amounts of data is collected. The trained machine learning algorithm may then use at least the signal strength received from the nodes within short-range wireless communication range (including the current controller node and the current connection node for the personal device if any of these two nodes are within short-range wireless communication range with the personal device to receive the broadcasts) and/or sensor data detected by a movement sensor of the personal device to determine the current position or location for the personal device.

In aspects, the technology disclosed relates to a method for a system comprising at least two or at least three network nodes configured to share data with the other nodes via an IP-based network and a plurality of personal devices each with Bluetooth communication capabilities, the method comprising the steps of:
a. establishing, by a connection node assigned to a personal device, a short-range wireless communication link between the connection node and the personal device, where the short-range wireless communication link is the only current communication link between the personal device and the network of nodes;
b. transmitting, from the personal device, data via the established short-range wireless communication link to the connection node; and
c. transmitting, from the personal device, short-range wireless broadcast messages, where the short-range wireless broadcast messages are transmitted while the personal device is transmitting data over and/or has the established short-range wireless communication link with the connection node assigned to the personal device.

In aspects, the technology disclosed relates to a method for a system comprising network nodes configured to share data with the other nodes via an IP-based network and a plurality of personal devices each with Bluetooth communication capabilities, the method comprising the steps of:
a. assigning, e.g. automatically by the nodes of the system, only one single controller node among the network nodes to have the sole responsibility for a personal device;
b. assigning, by the controller node, only one single connection node for the personal device, where the controller node is sole responsible for assigning connection node for the personal device;
c. establishing, by the assigned connection node, a short-range wireless communication connection link between the connection node and the personal device, where the short-range wireless communication connection link is the only current connection link between the personal device and the network of nodes;
d. transmitting, from the personal device, data via the established short-range wireless communication connection link to the connection node; and
e. transmitting, from the personal device, short-range wireless broadcast messages, where the short-range wireless broadcast messages are transmitted while the personal device is transmitting data over the established short-range wireless communication link with the connection node.

In aspects, the technology disclosed relates to a method for a system comprising at least two network nodes configured to share data with the other nodes via an IP-based network and a plurality of personal devices with Bluetooth communication capabilities, said method comprising:
a. assigning, e.g. by the nodes of the system using a common decision model which is known to all of the at least two network nodes, only one single controller node among the at least two network nodes to have the sole responsibility for said personal device;
b. assigning, by said assigned only one single controller node, only one single connection node for said personal device, wherein said controller node is sole responsible for assigning said only one single connection node to said personal device;
c. establishing, by said assigned only one single connection node, a Bluetooth connection between said only one single connection node and said personal device, wherein said Bluetooth connection is the only currently established Bluetooth connection from the network to said personal device;
d. transmitting, from said personal device, data via said established Bluetooth connection to said only one single connection node; and
e. transmitting, from said personal device, Bluetooth broadcast signals, wherein said Bluetooth broadcast signals are transmitted while said personal device has said established Bluetooth connection with said only one single connection node.

In embodiments, the technology disclosed relates to a method for a system comprising at least two network nodes and a plurality of personal devices with Bluetooth communication capabilities, said method comprising:
a. assigning, e.g. by the nodes of the system using a common decision model which is known to all of the at least two network nodes, only one single controller node among the at least two network nodes to have the sole responsibility for said personal device;
b. assigning, by said assigned only one single controller node, only one single connection node for said personal device, wherein said controller node is sole responsible for assigning said only one single connection node to said personal device;
c. establishing, by said assigned only one single connection node, a Bluetooth connection between said only one single connection node and said personal device, wherein said Bluetooth connection is the only current Bluetooth connection between the at least two network nodes and said personal device;
d. identifying, by said personal device, at least one event associated with said personal device or the user or wearer of said personal device;
e. transmitting, from said personal device, event data representing, or adapted to be used for identifying, said at least one event via said established Bluetooth connection to said only one single connection node;
f. transmitting or forwarding, from said connection node and following or in response to receiving said event data by said connection node, event data representing, or adapted to be used for identifying, said at least one event to said controller node;
g. transmitting, from said personal device, event data representing, or adapted to be used for identifying, the same at least one event in at least one Bluetooth broadcast message, wherein said at least one Bluetooth broadcast message is transmitted concurrently or in parallel with said transmission of event data via said established Bluetooth connection and while said personal device has said established Bluetooth connection with said only one single connection node;
h. receiving, by a plurality of network nodes within Bluetooth broadcast range with said personal device, said at least one Bluetooth broadcast message containing event data representing, or adapted to be used for identifying, said at least one event; and
i. transmitting, from at least one network node within Bluetooth broadcast range with said personal device, event data representing, or adapted to be used for identifying, said at least one event to said controller node.

In embodiments, the technology disclosed relates to a method for a system comprising at least two network nodes configured to share data with the other nodes via an IP-based network and a plurality of personal devices with Bluetooth communication capabilities, said method comprising:
a. assigning only one single controller node among the at least two network nodes to have the sole responsibility for said personal device;
b. assigning, by said assigned only one single controller node, only one single connection node for said personal device, wherein said controller node is sole responsible for assigning said only one single connection node to said personal device;
c. establishing, by said assigned only one single connection node, a Bluetooth connection between said only one single connection node and said personal device, wherein said Bluetooth connection is the only currently established Bluetooth connection between the at least two network nodes constituting the IP-based network and said personal device;
d. identifying, by said personal device, at least one occurred event associated with said personal device;
e. transmitting, from said personal device, event data representing said at least one occurred event via said established Bluetooth connection to said only one single connection node;
f. transmitting or forwarding, from said connection node and in response to receiving said event data by said connection node, event data representing said at least one occurred event to said controller node via said IP-based network;
g. transmitting, from said personal device, event data representing said same at least one occurred event in at least one Bluetooth broadcast message, wherein said at least one Bluetooth broadcast message is transmitted concurrently or in parallel with said transmission of event data via said established Bluetooth connection and while said personal device has said established Bluetooth connection with said only one single connection node;
h. receiving, by a plurality of network nodes within Bluetooth broadcast range with said personal device, said at least one Bluetooth broadcast message containing event data representing said same at least one occurred event; and
i. transmitting, from at least one network node within Bluetooth broadcast range with said personal device, event data representing said at least one occurred event to said controller node via said IP-based network.

In various embodiments, the concurrent or parallel transmissions of event data representing the same at least one event from the personal device via both the established Bluetooth connection and via at least one Bluetooth broadcast message are triggered by at least one event identified by the personal device.

In embodiments, the at least one event identified by the personal device and which triggers the parallel and redundant transmissions of event data may be sensor data sensed or measured by a sensor of the personal device or may be triggered by activating, by the individual or object carrying or wearing the personal device, an input means of the personal device such as the event of pressing of a button, e.g. an alarm button, on the personal device or an event identified by voice activation using a voice activation means of the personal device.

In embodiments, the sensed or measured sensor data that triggers the parallel and redundant transmissions of event data representing the at least one event may include that the personal device identifies that a sensed or measured sensor data exceeds or under-runs at least one predefined threshold value, e.g. a predefined threshold temperature value such as a predefined threshold body temperature value of the individual or object carrying or wearing the personal device.

In embodiments, the sensor which sensed or measured sensor data triggers the parallel or redundant transmissions of event data from the personal device may be a location or position detector or sensor, e.g. an RFID means, for determining the current position or location of the individual carrying or wearing the personal device. The sensed or measured sensor data that triggers the parallel and redundant transmissions of event data representing the at least one event may then include that the personal device identifies that the individual or object carrying or wearing the personal device has changed his/her location or position by receiving or exchanging, e.g. wirelessly receiving or exchanging location or position data, from/with a wireless transmitter or transceiver. The received or exchanged location or position data may then be used by the personal device to determine that the personal device is in a certain position or location, e.g. a certain room, of a facility in which a monitored individual may be residing, e.g. a nursing home, where there may be a private space for the monitored individual, including e.g. a bedroom, as well as public spaces that are shared by a plurality of monitored individuals. The parallel or redundant transmissions of event data may also be at least partly based on a timer of the personal device is indicating a certain movement pattern of the individual wearing the personal device, and/or at least partly based on a timer of the personal device is indicating that a certain predefined time period has lapsed since the individual activated an input means of the personal device, e.g. that a certain predefined time period has lapsed since the individual pressed a button of the personal device

In embodiments, the sensor which sensed or measured sensor data triggers the parallel or redundant transmissions of event data from the personal device may be a movement sensor such as an accelerometer for determining the movement speed and/or pattern of the individual carrying or wearing the personal device. The sensed or measured sensor data that triggers the parallel and redundant transmissions of event data representing the at least one event may then include that the personal device identifies that a sensed or measured sensor data exceeds or under-runs at least one predefined threshold value, e.g. a predefined threshold movement speed, a predefined threshold average movement distance per time unit and/or threshold movement pattern value of the individual or object carrying or wearing the personal device.

In embodiments, the sensor which sensed or measured sensor data triggers the parallel or redundant transmissions of event data from the personal device may be a health or medical sensor such a body temperature sensor for determining the body temperature of the wearer, or may include a blood pressure sensor, a heart rate sensor, a skin galvanometric level sensor etc.

In embodiments, the personal device may be a wristband and at least one sensor of the personal device may include at least one health or medical sensor adapted for obtaining sensor data for determining the medical and/or physical condition of the wearer of the wristband, e.g. a medical sensor configured to sense a characteristic of the user and generate a sensor output signal. The at least one health sensor, or medical sensor, may then include e.g. a temperature sensor for obtaining sensor data for determining the body temperature of the wearer, or may include a blood pressure sensor, a heart rate sensor, a skin galvanometric level sensor etc.

In embodiments, the technology disclosed relates to a method for determining the current position or location for a personal device of a system comprising at least two network nodes and a plurality of personal devices with Bluetooth communication capabilities, said method comprising:
a. assigning only one single controller node among the at least two network nodes to have the sole responsibility for said personal device;
b. assigning, by said assigned only one single controller node, only one single connection node for said personal device, wherein said controller node is sole responsible for assigning said only one single connection node to said personal device;
c. establishing, by said assigned only one single connection node, a Bluetooth connection between said only one single connection node and said personal device, wherein said Bluetooth connection is the only current Bluetooth connection between the at least two network nodes and said personal device;
d. transmitting, from said personal device, data via said established Bluetooth connection to said only one single connection node;
e. transmitting, from said personal device, data in at least one Bluetooth broadcast message, wherein said at least one Bluetooth broadcast message is transmitted concurrently or in parallel with said transmission of data via said established Bluetooth connection and while said personal device has said established Bluetooth connection with said only one single connection node;
f. receiving, by a plurality of network nodes within Bluetooth broadcast range with said personal device, said at least one Bluetooth broadcast message;
g. measuring, by said plurality of network nodes within Bluetooth broadcast range with said personal device, signal strength values for the received at least one Bluetooth broadcast message;
h. transmitting or forwarding, from the plurality of network nodes within Bluetooth broadcast range with said personal device, said measured signal strength values, or data representing or reflecting said measured signal strength values, to said controller node; and
i. determining, by said controller node, the current position or location for the personal device at least partly based on the received measured signal strength values separately received from the plurality of network nodes.

In embodiments, the technology disclosed relates to a method for determining the current position or location for a personal device of a system comprising at least two network nodes and a plurality of personal devices with Bluetooth communication capabilities, said method comprising:
a. assigning only one single controller node among the at least two network nodes to have the sole responsibility for said personal device;
b. assigning, by said assigned only one single controller node, only one single connection node for said personal device, wherein said controller node is sole responsible for assigning said only one single connection node to said personal device;
c. establishing, by said assigned only one single connection node, a Bluetooth connection between said only one single connection node and said personal device, wherein said Bluetooth connection is the only current Bluetooth connection between the at least two network nodes and said personal device;
d. transmitting, from said personal device, data via said established Bluetooth connection to said only one single connection node;
e. transmitting, from said personal device, data in at least one Bluetooth broadcast message, wherein said at least one Bluetooth broadcast message is transmitted concurrently or in parallel with said transmission of data via said established Bluetooth connection and while said personal device has said established Bluetooth connection with said only one single connection node;
f. receiving, by a plurality of network nodes within Bluetooth broadcast range with said personal device, said at least one Bluetooth broadcast message;
g. measuring, by said plurality of network nodes within Bluetooth broadcast range with said personal device, signal strength values for the received at least one Bluetooth broadcast message;
h. transmitting or forwarding, from the plurality of network nodes within Bluetooth broadcast range with said personal device, said measured signal strength values, or data representing or reflecting said measured signal strength values, to said controller node; and
j. determining, by said controller node or a locally-installed or remote processing arrangement communicatively coupled to the controller node, the current position or location for the personal device at least partly based on the received measured signal strength values separately received from the plurality of network nodes.

In embodiments, the technology disclosed relates to a method for changing connection node for a personal device of a system comprising at least two network nodes and a plurality of personal devices with Bluetooth communication capabilities, said method comprising:
a. assigning only one single controller node among the at least two network nodes to have the sole responsibility for said personal device;
b. assigning, by said assigned only one single controller node, only one single connection node for said personal device, wherein said controller node is sole responsible for assigning said only one single connection node to said personal device;
c. establishing, by said assigned only one single connection node, a Bluetooth connection between said only one single connection node and said personal device, wherein said Bluetooth connection is the only current Bluetooth connection between the at least two network nodes and said personal device;
d. transmitting, from said personal device, data via said established Bluetooth connection to said only one single connection node;
e. transmitting, from said personal device, data in at least one Bluetooth broadcast message, wherein said at least one Bluetooth broadcast message is transmitted concurrently or in parallel with said transmission of data via said established Bluetooth connection and while said personal device has said established Bluetooth connection with said only one single connection node;
f. receiving, by a plurality of network nodes within Bluetooth broadcast range with said personal device, said at least one Bluetooth broadcast message;
g. measuring, by said plurality of network nodes within Bluetooth broadcast range with said personal device, signal strength values for the received at least one Bluetooth broadcast message;
h. transmitting or forwarding, from the plurality of network nodes within Bluetooth broadcast range with said personal device, said measured signal strength values, or data representing or reflecting said measured signal strength values, to said controller node; and
k. determining, by said controller node or a locally-installed or remote processing arrangement communicatively coupled to the controller node, to change connection node, or assign a new connection node, for said personal device, wherein said change of connection node is at least partly based on the received measured signal strength values separately received from the plurality of network nodes, and wherein said new connection node is among said plurality of network nodes and is the new only one single connection node for said personal device.

In aspects, the technology disclosed relates to a system including a network comprising at least two network nodes configured to share data with the other nodes via an IP-based network and a plurality of personal devices with Bluetooth communication capabilities, said plurality of personal devices are each assigned only one controller node among the at least two network nodes, wherein the only one controller node is responsible for assigning only one connection node among the at least two network nodes to be responsible for establishing a Bluetooth connection with the personal device the controller node is responsible for, and wherein said plurality of personal devices are each configured to be transmitting Bluetooth broadcast signals while having an established Bluetooth connection with their respectively assigned only one connection node.

In embodiments, the only one controller node assigned to the respective personal device is configured to assign and dynamically change which of the at least two network nodes is currently the only one connection node for their respective personal devices at least partly based on at least one of contents and received signal characteristics of at least one Bluetooth broadcast signal transmitted from the personal device and which is received by a plurality of other nodes than the controller node.

In embodiments, the only one controller node assigned to the respective personal device is configured to receive data via said IP-based network from a plurality of other nodes than the controller node, and wherein said data received via said IP-based network include data indicating the measured signal strength values of Bluetooth broadcast signals transmitted by the respective personal device and which are received and measured by the plurality of other nodes.

In embodiments, the only one controller node assigned to the respective personal device is further configured to change the only one connection node for the personal devices the only one controller node is responsible for at least partly based on said data indicating the measured signal strength values of Bluetooth broadcast signals transmitted by the personal device and which are received and measured by the plurality of other nodes.

In embodiments, the only one controller node assigned to the respective personal device is further configured to determine the current position of the personal device it is responsible for based on said data indicating the measured signal strength values of Bluetooth broadcast signals transmitted by the respective personal device and which are received and measured by the plurality of other nodes.

In embodiments, the system further comprises a locally-installed or remote processing arrangement such as a backend system and network configured to receive data indicating measured Bluetooth broadcast signal strength values for a personal device from the only one controller node responsible for the personal device, and wherein said locally-installed or remote processing arrangement, .e.g. backend system and network, is further configured to determine the current position for the personal device based on the received Bluetooth broadcast signal strength values for the personal device and return said determined position to the only one controller node responsible for the personal device.

In embodiments, each of said plurality of personal devices is configured to transmit Bluetooth broadcast signals including alarm data in response to a trigger event determined by the respective personal device to have occurred, and wherein said alarm data is adapted to indicate the occurrence of said trigger event to any node receiving the Bluetooth broadcast signal and is transmitted by the respective personal device while having an established Bluetooth connection with their respectively assigned only one connection node.

In embodiments, each of said plurality of personal devices is further configured to, in addition, transmit data including redundant alarm data in response to the determined occurrence of the trigger event via the established Bluetooth connection with their respectively assigned only one connection node, and wherein the respectively assigned only one connection node is configured to, in response to receiving said alarm data via said established Bluetooth connection, at least one of transmit and forward data including alarm data via said IP-based network to the respectively assigned only one controller node, where said alarm data is adapted to indicate to the only one controller node that the trigger event has occurred.

In embodiments, said system comprises at least one mobile communication device and a backend system and network configured to receive alarm data indicating the occurred trigger event from the only one controller node responsible for the personal device which determined that the trigger event occurred, and wherein said backend system and network is further configured to transmit alarm data to the at least one mobile communication device in response to receiving said alarm data from the only one controller node.

In embodiments, the local or remote processing arrangement, e.g. backend system and network, is further configured to transmit said alarm data to the at least one mobile communication device via a network separate from said IP-based network used by the at least two network nodes to exchange data.

In embodiments, said trigger event is the activation of an input means on the respective personal device.

In embodiments, said personal device is a wristband and said trigger event is the action of pressing a button on the wristband or activating a voice activation means of the personal device.

According to certain aspects and embodiments, the assignment of roles for the network nodes is associated with a specific individual personal device and is done on two levels, a control level through the assignment of a controller node and a connect level through the assignment of a connection node. The role assigned to a network node may be dynamically changed based on data obtained by the personal devices and/or the node network.

The system, e.g. monitoring system, comprising the node network of the technology disclosed is highly scalable and provides reliable and fast responses to obtained sensor data, event data and inputs from the monitored individuals, yet is robust in that each of the personal devices always has a reliable short-range wireless connection to the node network which is not disturbed or compete for short-range wireless bandwidth with other devices connected to the same node of the node network.

According to certain aspects and embodiments, the system and methods of the technology disclosed provides a scalable solution in the dynamic reconfiguration of the node networks on two levels, yet is robust and reliable in that each of the personal devices always is provided a reliable short-range wireless connection to the network. In embodiments, this is achieved by assigning a controller node to each personal device which is responsible for assigning a connection node to the personal device and dynamically changing connection node for the personal device, where the connection node is responsible for establishing a short-range wireless connection with the personal device.

According to aspects of the technology disclosed, the other network nodes shares or forwards any received or obtained data or information, e.g. status data, sensor data, event data or input data, which is received from or associated with a certain personal device to the controller node for the personal device. This allows for redundancy and, thus, safer and more accurate decision-making by the controller node, as well as provides faster responses to events, e.g. the pressing of a button on the personal device, and obtained sensor data in that all data or information associated with a certain personal device is quickly available to the one controller node having the sole responsibility for taking actions and making decisions on behalf of the personal device, e.g. fast responses in terms of making a decision to set an alert, change a health state and/or change connection node for the personal devices.

According to aspects of the technology disclosed, there is only one controller node for each personal device and the system is configured so that each of the other network nodes shares or forwards any received or obtained data or information, e.g. status data, sensor data, event data or input data, associated with a certain personal device to the controller node for the personal device. This provides for redundancy and, thus, safer and more accurate decision-making by the controller node as well as faster responses to events and obtained data in that all data or information associated with a certain personal device is received by the one controller node having the sole responsibility for making decisions and taking actions on behalf of the personal device.

In aspects, the technology disclosed proposes a system and node network for dynamically assigning roles to the nodes of the network on two levels, a control level and connect level. Each of the personal device may then be assigned only one network node, on the control level, to be controller node for the personal device and only one network node, on the connect level, to be the connection node for the personal device. The assigned controller is then responsible for dynamically assigning only one connection node for the personal device at a time. The controller node assigned to a personal device may be assigned according to a common decision model known to and used by the at least two network nodes and following the sharing or distribution of data associated with the personal device. In embodiments, the data shared between the nodes may then be used as input values to the common decision model for dynamically changing controller node for the personal device.

According to aspects of the technology disclosed, there is only one connection node for establishing a short-range wireless connection with the personal device and the system is configured so that the controller node for the personal device has the sole responsibility for changing connection node for the personal devices. This provides for a fast response yet robust and scalable solution for monitoring personal devices, e.g. wristbands, necklaces or clips carried or worn by individuals moving between locations or rooms of a facility such as patients in a nursing home. The established short-range wireless connection is then the only connection between the network and the personal device. According to this embodiment, the personal device may then still transmit short-range wireless broadcast signals to provide the other network nodes within short-range wireless communication range with status data, obtained sensor data, event data and/or input data associated with the personal device.

In certain embodiments of the technology disclosed, the system is configured so that the connection node assigned to a personal device is never the assigned controller node for the personal device.

In embodiments of the technology disclosed, the system is configured so that the connection node assigned to a personal device is never the currently assigned controller node for the personal device, yet the controller node and connection node assigned to a personal device may be dynamically changed so that a node that previously was connection node for a personal device may be the controller node for the personal device, and vice versa.

According to aspects of the technology disclosed, the wireless transmissions of data between the personal devices and the nodes of the network of nodes are thereby separated from the transmissions, e.g. wireless transmission, of data between the nodes of the node network. The data shared between the nodes of the network may then be transmitted over an IP-based network, e.g. WiFi or Ethernet, and the data transmitted from the personal devices to the node network and the data exchanged between the personal devices and the individual nodes of the network may be transmitted via short-range wireless communication, e.g. using a short-range wireless communication protocol such as Bluetooth or other protocols providing similar short-range communication range.

According to aspects of the technology disclosed, the data transmitted from the personal devices to the node network and between the personal devices and the network nodes may then contain sensor data or event data obtained and transmitted by the personal devices and then received by at least one network node within short-range wireless communication range. The transmission of data between the at last three network nodes may include at least one of the transmission of data associated with a certain personal device which is transmitted or forwarded from one of the nodes to the assigned controller node for the personal device, transmission of data which is shared between the nodes prior to assigning a node to be the controller node for a personal device and the transmission of data from the controller node to inform and/or instruct a (new) connection node which is assigned, e.g. dynamically assigned, by the controller node to be the connection node for a personal device and which is assigned to establish a short-range wireless connection with the personal device.

The technology disclosed provides a more scalable system, e.g. monitoring system, comprising a node network that does not rely on the presence of other personal devices within short-range communication range for making decisions on behalf of a personal device and, since the personal devices connected to the node network are not sharing their short-range wireless communication interface to the node network with short-range wireless connections to other personal devices, is less dependent on the currently available short-range wireless bandwidth. The technology disclosed comprising a node network where the nodes are transmitting obtained data associated with a certain personal device to the controller node responsible for the personal is therefore less dependent on the current number of other short-range wireless connections to the communication interface of the personal devices.

According to aspects, the controller node having the sole responsibility for a certain personal device is responsible for assigning and dynamically changing which of the at least two network nodes is the connection node for the personal device. The controller node may then decide to change connection node for a personal device the controller node is responsible for at least partly based on data received from the other nodes, e.g. measured signal strengths of short-range broadcast signals or messages transmitted by the personal device and which are received by any of the other nodes which are within short-range communication range with the personal device. The controller node may thereby see to that the personal device always has the best possible or available short-range wireless connection, e.g. Bluetooth connection, to the network of nodes which in turn may be communicatively coupled to a backend system. This provides for a scalable system which responds quickly to events, obtained sensor data and the movements of the individuals in that data is shared between the nodes, yet is robust in that each of the personal devices is continuously and always provided with a reliable short-range wireless connection to the network.

The dynamic assignment of connection node by the controller node by taking into account at least one of signal strengths of broadcast signals measured by the nodes within short-range wireless communication range and the number of current short-range connections to the other network nodes also provides improved load-balancing in the network. The controller node, or a local or remote processing arrangement such as a backend system communicatively coupled to the controller node, may further use a more advanced load balancing algorithm to further improve the load-balancing in the network where the load-balancing algorithm may consider at least one of the number of personal devices each of the network nodes is controller node for, the number of short-range wireless connections in a certain location or geographical sub-area of the facility and the number of personal devices in a certain location or geographical sub-area of the facility. In aspects, the technology disclosed relates to a system comprising a node network which is self-organizing by assigning a controller node to each personal device within short-range communication range with at least one of the nodes of the network.

In embodiments, the assignment of a controller node may use a common decision model which is known to the nodes of the node network where the decision model may, or may not, use input or parameter data values obtained from the personal devices and/or other nodes of the network to determine which of the at least two nodes is going to be the controller node. The input or parameter data values may change the output from the use of the common decision model and the determining of which of the network nodes is going to be the controller node for a personal device. In some embodiments, the controller node assigned to a certain personal device may be dynamically changed at least partly based on obtained input or parameter data values.

In embodiments, the assignment, by the controller node, of a connection node may be at least partly based on signal strengths of short-range wireless broadcast signals which are measured by a plurality of network nodes within short-range communication range with the personal device transmitting the short-range wireless broadcast signals. The assignment of connection node by the controller node may further be based on or use a load-balancing algorithm which may consider at least one of the number of personal devices each of the network nodes is controller node for, the number of short-range wireless connections in a certain location or geographical sub-area of the facility and the number of personal devices in a certain location or geographical sub-area of the facility.

In embodiments, the technology disclosed relates to a personal device, a system comprising at least two network nodes and methods in a system comprising a Self-Organizing Network (SON) and a plurality of personal device, e.g. a plurality of wristbands worn by individuals such as patients.

In aspects, the technology disclosed relates to methods for dynamically assigning roles to network nodes, a system for monitoring a plurality of personal devices and a portable personal device with short-range communication capability for communicating with network nodes.

The system, e.g. monitoring system, may comprise a memory configured to store obtained or received data associated with the individual personal devices, e.g. data detected or registered by a sensor of the individual personal device or measurement data provided by network nodes of the system. The system may further comprise a data processing unit and computer program code control logics.

In aspects and certain embodiments, the technology disclosed relates to a system including a network comprising at least two network nodes configured to transmit data to the other nodes via the network and a plurality of personal devices with short-range wireless communication capabilities for communicating with the at least two network nodes. The plurality of personal devices are each assigned only one controller node among the at least two network nodes, and the respective controller node is responsible for assigning a connection node among the at least two network nodes to establish a short-range wireless communication connection with a personal device the controller node is responsible for.

In embodiments, the system, e.g. monitoring system, is configured so that each of said plurality of personal devices is assigned only one controller node which has the sole responsibility for the personal device.

In embodiments, said system is configured so that each or at least one of the at least two network nodes may be the assigned sole controller node for a plurality of personal devices.

In embodiments, said system including the at least two network nodes is configured to use a common decision model which is known to each of the at least two nodes for determining which of the at least two nodes is going to be the controller node assigned to the respective personal device.

In embodiments, said common decision model use input values or parameters which are continuously and dynamically changed at least partly based on data distributed and shared between the network nodes, thereby allowing for the determining of which of the nodes among the at least two nodes is going to be the controller node for the personal device to be dynamically changed at least partly on data distributed and shared between the network nodes. The network for distributing and sharing data between the network nodes is typically an IP-based network providing for improved scalability, e.g. a WiFi or Ethernet.

In embodiments, said system is configured so that each assigned network node to be connection node for a personal device only has one established short-range wireless communication connection with the node network at a time.

In embodiments, said plurality of personal devices are each configured to be transmitting short-range wireless broadcast signals while having an established short-range wireless communication connection with their respective connection node.

In embodiments, the only one controller node assigned to the respective personal device is configured to assign and dynamically change which of the at least two network nodes is currently the only one connection node for the personal device at least partly based on at least one of contents and received signal strengths of at least one short-range wireless broadcast signal transmitted from the personal device and which is received by a plurality of other nodes than the controller node.

In embodiments, the only one controller node assigned to the respective personal device is configured to receive data from a plurality of other nodes than the controller node via an IP-based network connecting the at least two network nodes, and wherein said data received via said IP-based network include data indicating the measured signal strength values of short-range wireless broadcast signals transmitted by the respective personal device and which are received and measured by the plurality of other nodes.

In embodiments, the only one controller node assigned to the respective personal device is further configured to determine a position or location for the personal device and/or change the only one connection node for the personal devices the only one controller node is responsible for at least partly based on said data indicating the measured signal strength values of short-range wireless broadcast signals transmitted by the respective personal device and which are received and measured by the plurality of other nodes and, in addition, at least partly based on signal strengths measured by the current controller node and the current connection node for the personal device if any of these nodes are within short-range wireless communication range with the personal device to receive the broadcasts.

In embodiments, the network comprises at least two network nodes configured to transmit data to the other nodes via the network is an IP-based network separated from and using a different communication protocol than used for the short-range wireless connections established between the personal devices and the network nodes. The short-range wireless capabilities of the plurality of personal devices for communicating with the at least two network nodes may then typically be Bluetooth communication capabilities, or similar short-range wireless capabilities.

In embodiments, said plurality of personal devices may include a plurality of wristbands, necklaces or clips, each may be provided with a sensor or an input means and worn by an individual such a patient of a nursing home.

In aspects, the technology disclosed relates to a method in a system, e.g. a monitoring system, including a network comprising at least two network nodes and a plurality of personal devices with short-range wireless communication capabilities, said method comprising:
a. assigning only one node among the at least two network nodes to be the controller node having the sole responsibility for a particular personal device;
b. assigning, by said controller node, a connection node for establishing a short-range wireless communication connection between said connection node and said personal device;
c. establishing, by said assigned connection node, a short-range wireless communication connection with said personal device;
d. transmitting, from said personal device and via said established short-range wireless communication connection, a message to said connection node;
e. receiving, by said connection node, said message, wherein said message contains data including at least one of status data, sensor data and event data associated with said personal device;
f. transmitting, from the assigned connection node to the sole controller node for said personal device, said received message or a message including data corresponding to, or at least partly based on, said at least one of status data, sensor data and event data associated with said personal device; and
g. transmitting, from said controller node to at least one of at least one of the other at least two network nodes and a backend system, a message containing data adapted to be used for at least one of controlling and monitoring the personal device, wherein said message is at least partly based on said data corresponding to, or at least partly based on, said at least one of status data, sensor data and event data associated with said personal device.

In embodiments, the short-range wireless communication connection is the only currently established communication connection between the network nodes and said personal device.

In embodiments, each of the plurality of personal devices is assigned only one of at least three network nodes to be the controller node for the respective personal device.

In embodiments, each of the plurality of personal devices is assigned, by their respective controller node, only one of at least three network nodes to be the connection node for the personal device.

In embodiments, each of the at least three network nodes has no more than one established short-range wireless connection with a personal device at a time.

In embodiments, the above method further comprises distributing or sharing data between at least three network nodes via an IP-based network not using the same short-range wireless communication protocols used by the personal devices for communicating with the node network. The distributing or sharing of data between the nodes is preceding the assignment of the controller node, and the data distributed or shared between at least three network nodes is further used for determining which of at least three nodes is going to be the controller node for the personal device.

In embodiments, the distributing or sharing of data between the nodes is preceding the assignment of the controller node, and the data distributed or shared between at least three network nodes is used for determining which of the at least three nodes is going to be the controller node for the personal device.

In embodiments, the method further comprises determining which of at least three nodes is going to be the controller node for the personal device by using a common decision model which is known to each of the at least three nodes.

In embodiments, the common decision model use input values or parameters which are continuously and dynamically changed at least partly based on data distributed and shared between the network nodes, thereby allowing for the determining of which of the nodes among at least three nodes is going to be the controller node for the personal device to be dynamically changed at least partly on data distributed and shared between the network nodes.

In embodiments, the method further comprises forwarding, by at least a plurality of nodes within short-range wireless communication range with the personal device, at least a portion of the contents of said short-range wireless broadcast signals to the other network nodes. The at least a portion of the contents of said broadcast signals may then be used by the network of nodes as input values to the for determining which of at least three nodes is going to be the controller node for the personal device.

In embodiments, the method further comprises forwarding, by at least a plurality nodes within short-range wireless communication range with the personal device, at least a portion of the contents of said short-range wireless broadcast signals to the controller node. The contents of said broadcast signals may then be adapted to be used by the controller node, or by a local or remote processing arrangement, for determining the position or location of the personal device for determining which of at least three nodes is to be assigned connection node for the personal device.

The at least one sensor of the personal device may include an accelerometer adapted to detect the movements of the portable personal device, e.g. the acceleration and movement pattern of the portable personal device.

In certain embodiments, the technology disclosed relates to methods and a system for monitoring an individual within predetermined facilities by identifying an event associated with a personal device carried or worn by the individual, e.g. with the purpose of determining whether there is a need for setting an alert. The system may comprise at least three network nodes and a plurality of personal devices each carried or worn by the respective monitored individual, where each of the plurality of personal devices comprises at least one sensor and has short-range communication capability, e.g. Bluetooth communication capability, with which the respective personal device is arranged to communicate with the network nodes. The personal device may also comprise a storage means and at least one processing device.

In embodiments, the personal device may be a wristband and the at least one sensor of the personal device may comprise at least one medical sensor, or health sensor, adapted for obtaining sensor data for determining the medical and/or physical condition of the wearer of the wristband, e.g. a medical sensor configured to sense a characteristic of the user and generate a sensor output signal.

In embodiments, the personal device may be a wristband and the at least one sensor of the personal device may comprise at least one health sensor adapted for obtaining sensor data for determining the medical and/or physical condition of the wearer of the wristband, e.g. a medical sensor configured to sense a characteristic of the user and generate a sensor output signal. The at least one health sensor, or medical sensor, may then include e.g. a body temperature sensor for obtaining sensor data for determining the body temperature of the wearer, or may include a blood pressure sensor, a heart rate sensor, a skin galvanometric level sensor etc.

In embodiments, the at least one processing device of the personal device may be arranged to receive sensor data from the at least one sensor in the personal device and determine whether the received sensor data indicates an alert state, e.g. a specific alert state among a plurality of predetermined alert states.

In certain embodiments, the alert states may comprise at least a FALL alert state to be used if a probable fall has been detected for the monitored individual and an OUT OF BED alert state to be used if it has been detected that the monitored individual is probably getting out of bed.

According to certain aspects of the technology disclosed, personal devices communicate with nodes in the node network using Bluetooth, e.g. Bluetooth broadcasts or via an established Bluetooth connection, whereas the communication between the network nodes takes place using the IP addresses of the nodes (e.g. via Wi-Fi). The network of nodes may then be self-organizing in that the nodes of the network continuously and/or automatically assigns roles to the individual nodes, where the assignment of a role is preceded by, and at least partly based on, data distributed and/or shared between the network nodes. The distributed and/or shared data is obtained by the other network nodes and is associated with the personal device to which the assignment of a role concerns.

The personal devices may broadcast their own universal unique identifiers (UUIDs) and seek to establish connections with Bluetooth devices within its Bluetooth communication range. The network nodes may be configured to store the UUIDs of all personal devices detected in their memories. The connection node assigned, by the controller node, to a certain personal device may then establish a Bluetooth connection with the personal device by transmitting a connection request via Bluetooth to the personal device where the connection request may include the unique identifier (UUID) of the personal device.

According to aspects, the network nodes may be self-organizing in that the assignment of the one controller node having the sole responsibility for a specific personal device is a distributed decision which is preceded by at least one of distributing and sharing data between the network nodes using the IP-based network and by using a predetermined and common decision model with dynamic input values/parameters which are changed with the data distributed and shared between the network nodes.

According to embodiments, the network nodes may be self-organizing in that the decision to change controller node for a specific personal device is a distributed decision which is preceded by at least one of distributing and sharing of data between the network nodes using the IP-based network and by using a predetermined and common decision model with dynamic input values/parameters which are changed with the data distributed and shared between the network nodes.

According to aspects of the technology disclosed, personal devices transmits data to the nodes in the node network using Bluetooth, e.g. via Bluetooth broadcasts and an established Bluetooth connection with one of the nodes, and the transmission of data, e.g. data associated with the individual personal device, takes place using their IP addresses, e.g. data received from a certain personal device or associated with a certain personal device may be shared between network nodes via the IP-based network connecting the network nodes.

According to aspects, the personal devices are not part of the IP-based network connecting the but are only configured to transmit data and/or communicate with the network nodes via Bluetooth, e.g. using an established Bluetooth connection with one of the nodes and transmitting Bluetooth broadcast signals which may be received (and measured) by any network node within Bluetooth communication range from the personal device. In embodiments, the technology disclosed relates to a method for a system including a network, e.g. a Self-Organizing Network (SON), comprising at least three network nodes configured to share data with the other nodes via an IP-based network, and a plurality of personal devices with Bluetooth communication capabilities, the method comprising:
a. assigning only one controller node among the at least three network nodes to have the sole responsibility for a personal device;
b. assigning, by said assigned controller node, a connection node for the same personal device, where controller node is responsible for assigning said connection node to said personal device;
c. establishing, by the assigned connection node, a Bluetooth connection between the connection node and the personal device; and
d. transmitting, from the personal device, data via the established Bluetooth connection to the connection node;

In embodiments, the technology disclosed relates to a method for a system including a network, e.g. a Self-Organizing Network (SON), comprising at least three network nodes configured to share data with the other nodes via an IP-based network, and a plurality of personal devices with Bluetooth communication capabilities, the method comprising:
a. assigning only one controller node among the at least three network nodes to have the sole responsibility for a personal device, wherein said step of assigning the controller node is preceded by the sharing of data between the at least three nodes via the IP-based network;
b. assigning, by said assigned controller node, a connection node for the same personal device, wherein said controller node is responsible for assigning said connection node to said personal device;
c. establishing, by the assigned connection node, a Bluetooth connection between the connection node and the personal device; and
d. transmitting, from the personal device, data via the established Bluetooth connection to the connection node;

In embodiments, the technology disclosed relates to a method for a system including a network, e.g. a Self-Organizing Network (SON), comprising at least three network nodes configured to share data with the other nodes via an IP-based network, and a plurality of personal devices with Bluetooth communication capabilities, the method comprising:
a. assigning only one controller node among the at least three network nodes to have the sole responsibility for a personal device, wherein said only one controller node for said personal device is assigned by at least one of sharing data between the at least three network node and using a predetermined and common decision model with dynamic input values/parameters;
b. receiving, by a plurality of the at least three network nodes, Bluetooth broadcast data from said personal device;
c. forwarding, by said plurality of the at least three network nodes via the IP-based network and to said assigned only one controller node for said personal device, at least one of said received Bluetooth broadcast data, data or information corresponding to said received Bluetooth broadcast data and measured signal strength values for said received Bluetooth broadcast data;
d. assigning, by said assigned only one controller node, a connection node for said personal device, wherein said controller node is sole responsible for assigning connection node to said personal device, and wherein said assigning of connection node by said controller node is at least partly based on said at least one of said received Bluetooth broadcast data, data or information corresponding to said received Bluetooth broadcast data and measured signal strength values for said received Bluetooth broadcast data;
e. establishing, by said assigned only one connection node, a Bluetooth connection between the connection node and said personal device; and
f. transmitting, from said personal device, data via the established Bluetooth connection to the connection node;

In embodiments, the above method includes that the at least three network nodes (may be e.g. at least 5, 10, 20 or more network nodes) share data or information between each other via the IP-based network, e.g. an Ethernet or Wireless Fidelity (WiFi) network. The advantages of having the network nodes sharing data via an IP-based network whereas the personal devices connect to the network via Bluetooth connections include that the data may be distributed between

In embodiments, the assignment of controller node for a personal device may then be performed by distributing data or information received from or associated with the personal device to all of the at least three network nodes prior to deciding which of the at least three network nodes is going to be the controller node for the personal device. According to embodiments, the (distributed) decision between the at least three network nodes as to which of the at least three network nodes will be the controller node, e.g. new controller node, for the personal device is made according to a predetermined scoring system which is known to all of the at least three network nodes, where the scoring system may use dynamic variable input values. As an example, the at least three network nodes, e.g. constituting a Self-Organizing Network, may use a predetermined decision model, e.g. distributed consensus model. In various embodiments, the predetermined decision model may define a static hierarchy for the nodes or may have dynamic input values/parameters, where the input values/parameters may include, for example, data related to load balancing between different parts of the network and an assessment of the current traffic load distribution between the at least three nodes of the IP-based network, obtained (current) position data and/or signal strength values for personal devices broadcasting and/or having an established Bluetooth connection to one of the at least three network nodes, the number of personal devices each of the at least three network nodes is currently controller node for and the number of personal devices each of the at least three network nodes is currently connection node for.

In embodiments, the above method includes that the plurality of personal devices (may be e.g. at least 5, 10, 20, 50 or more personal devices) all have Bluetooth communication capabilities and all transmit Bluetooth broadcast messages/signals which may be received by any of the at least three network nodes (may be e.g. at least 5, 10, 20 or more network nodes) within Bluetooth communication range.

In embodiments, the above method includes that said Bluetooth connection is the only currently established Bluetooth connection from the network to said personal device and the connection node is the only network node having an established connection link with the personal device.

In embodiments, the technology disclosed relates to a method for a system including a network, e.g. a Self-Organizing Network (SON), comprising at least three network nodes configured to share data with the other nodes via an IP-based network and a plurality of personal devices with Bluetooth communication capabilities, the method comprising:
a. assigning only one controller node among the at least three network nodes to have the sole responsibility for a personal device among said plurality of personal devices;
b. assigning, by said assigned controller node, only one connection node for said personal device, wherein said controller node is sole responsible for assigning said only one connection node to said personal device;
c. establishing, by said assigned only one connection node, a Bluetooth connection between said only one connection node and said personal device, wherein said Bluetooth connection is the only currently established Bluetooth connection from the network to said personal device; and
d. transmitting, from said personal device, data via said established Bluetooth connection to said only one connection node;

In embodiments, the technology disclosed relates to a method for a system including a network, e.g. a Self-Organizing Network (SON), comprising at least three network nodes configured to share data with the other nodes via an IP-based network, e.g. an Ethernet or WiFi network, and a plurality of personal devices with Bluetooth communication capabilities, the method comprising:
a. assigning only one controller node among the at least three network nodes to have the sole responsibility for a personal device;
b. assigning, by the assigned controller node, a connection node for the same personal device, where the controller node is the only node responsible for assigning the connection node to the personal device;
c. establishing, by the assigned connection node, a Bluetooth connection between the connection node and the personal device;
d. transmitting, from the personal device, data via the established Bluetooth connection to the connection node; and
e. forwarding, by the connection node and via the IP-based network, the data received from the personal device to the assigned controller node for the personal device.

In embodiments, the technology disclosed relates to a method for assigning a connection node for a personal device in a Self-Organizing Network (SON) comprising at least three network nodes configured to share data with the other nodes via an IP-based network, e.g. an Ethernet or WiFi network, and a plurality of personal devices with Bluetooth communication capabilities, the method comprising:
a. assigning one of the at least three network nodes to be the controller node with sole responsibility for a personal device;
b. transmitting, by the personal device not having an established connection with any of the at least three network nodes, Bluetooth broadcast messages/signals;
c. receiving, by a plurality of the at least three network nodes within Bluetooth communication range, at least one Bluetooth broadcast message/signal from the personal device, where the at least one Bluetooth broadcast message/signal contains unique identification data for the personal device;
d. forwarding, from said plurality of nodes to the assigned controller node via the IP-based network, position data for the personal device and/or measured signal strength values for the personal device, wherein the position data and/or measured signal strength values are obtained through said plurality of nodes receiving said at least one Bluetooth broadcast message/signal;
e. determining, by the node assigned to be the controller node with sole responsibility for the personal device, to assign only one node among the at least three network nodes to be the sole connection node for the personal device, wherein the determining by the controller node of which node will be the connection node for the personal device is at least partly based said position data and/or measured signal strength values obtained through said plurality of nodes receiving said at least one Bluetooth broadcast message/signal; and
f. establishing, by the node determined by the controller node to be connection node for the personal device, a Bluetooth connection with the personal device.

In embodiments, the technology disclosed relates to a method for changing the connection node for establishing a Bluetooth connection with a certain personal device in a Self-Organizing Network (SON) comprising at least three network nodes configured to share data with the other nodes via an IP-based network, e.g. an Ethernet or WiFi network, and a plurality of personal devices with Bluetooth communication capabilities, the method comprising:
a. assigning only one controller node among the at least three network nodes to have the sole responsibility for a personal device;
b. assigning, by the assigned controller node, a connection node for the same personal device, where the controller node is the only node responsible for assigning the connection node to the personal device;
c. establishing, by the assigned connection node, a Bluetooth connection between the connection node and the personal device;
d. transmitting, from the personal device, data via the established Bluetooth connection to the connection node;
e. forwarding, by the connection node and via the IP-based network, the data received from the personal device (via the established Bluetooth connection) to the assigned controller node for the personal device;
f. transmitting, by said personal device, Bluetooth broadcast messages/signals, where the Bluetooth broadcast messages/signals are received by any of the at least three node which is within Bluetooth communication range; and
g. determining, by said controller node the personal device, a position and/or location for the personal device, where the determining of the position and/or location is at least partly based on measured signal strength values for Bluetooth broadcast messages/signals received by other nodes than the controller node and the current connection node.

In aspects, the technology disclosed relates to system comprising at least three network nodes and a plurality of personal devices, where each of the personal devices is assigned/having only one controller node and only one connection node at a time. The controller node for a personal device may then be assigned by sharing data between the nodes of the network and using a predetermined decision model, e.g. a distributed consensus model which is known to all of the at least three network nodes. In embodiments, the predetermined decision model may have dynamic input values/parameters, where the input values/parameters may include, for example, data related to load balancing between different parts of the network and an assessment of the current traffic load distribution between the at least three nodes of the IP-based network.

In aspects, the technology disclosed relates to a method in a system, e.g. a monitoring system, including a network comprising at least three network nodes configured to share data with the other nodes via an IP-based network and a plurality of personal devices with short-range wireless communication capabilities, the method comprising:
a. assigning only one controller node among the at least three network nodes to have the sole responsibility for a personal device;
b. assigning, by said controller node, only one connection node for said personal device, wherein said controller node is sole responsible for assigning said only one connection node to said personal device;
c. establishing, by said assigned only one connection node, a short-range wireless communication connection between said only one connection node and said personal device, wherein said short-range wireless communication connection is the only currently established connection from the network to said personal device;
d. transmitting, from said personal device, data via said established short-range wireless communication connection to said only one connection node; and
e. transmitting, from said personal device, short-range wireless broadcast signals, wherein said short-range wireless broadcast signals are transmitted while said personal device has said established Bluetooth connection with said only one connection node.

In embodiments, the method according to the technology disclosed is comprising:
a. receiving, by a plurality of nodes other than the only one controller node, said Bluetooth broadcast signals;
b. measuring, by each of said plurality of nodes, signal strengths values of said received Bluetooth broadcast signals;
c. transmitting, from each of said plurality of nodes and in response to receiving said Bluetooth broadcast signals, data to said only one controller node via said IP-based network, wherein said data is adapted to indicate said measured signal strengths values of said Bluetooth broadcast signals;
d. receiving, by said only one controller node and via said IP-based network, said transmitted data indicating said measured signal strengths values; and
e. determining, by said only one controller node, to change the only one connection node for said personal device at least partly based on said received data indicating said measured broadcast signal strengths values, wherein said new only one connection node for said personal device is either the assigned only one controller node or one or one of the plurality of nodes other than the controller node and is sole responsible for establishing a new Bluetooth connection to said personal device, wherein said new Bluetooth connection will be the only currently established Bluetooth connection from the network to said personal device.

In embodiments, the method according to the technology disclosed is comprising:
a. determining, by said only one controller node, the current position of said personal device based on said received data indicating said Bluetooth broadcast signal strengths values measured by said plurality of nodes.

In embodiments, the method according to the technology disclosed is comprising:
a. determining, by said personal device, that a trigger event has occurred, wherein said
b. wherein each of said plurality of personal devices is configured to transmit Bluetooth broadcast signals including alarm data in response to a trigger event determined by the respective personal device to have occurred, and wherein said alarm data is adapted to indicate the occurrence of said trigger event to any node receiving the Bluetooth broadcast signal and is transmitted by the respective personal device while having an established Bluetooth connection with their respectively assigned only one connection node.

In embodiments, the system comprises at least one mobile communication device and a backend system and network configured to receive alarm data indicating the occurred trigger event from the only one controller node responsible for the personal device which determined that the trigger event occurred, and wherein said backend system and network is further configured to transmit alarm data to the at least one mobile communication device in response to receiving said alarm data from the only one controller node.

In embodiments, the backend system and network is further configured to transmit said alarm data to at least one mobile device, e.g. at least one mobile device of a staff member of the facility, via a network separate from the IP-based network used by the at least three network nodes to exchange data.

In embodiments, the trigger event is the activation of an input means on the respective personal device.

In embodiments, the personal device is a wristband and said trigger event is the action of pressing a button on the wristband or activating a voice activation means of the personal device.

In embodiments, the only one controller node assigned to the respective personal device is configured to receive data via the network, e.g. an IP-based network, from a plurality of other nodes than the controller node. The data received via the IP-based network may then include data indicating the measured signal strength values of Bluetooth broadcast signals transmitted by the respective personal device and which are received and measured by the respective node of the plurality of other nodes.

In embodiments, the only one controller node assigned to the respective personal device is further configured to determine the current position and/or location of the personal device it is responsible for based on said data indicating the measured signal strength values of Bluetooth broadcast signals transmitted by the respective personal device and which are received and measured by the plurality of other nodes.

In embodiments, the system, e.g. monitoring system, further comprises a local or remote processing arrangement such as a backend system and network configured to receive data indicating measured Bluetooth broadcast signal strength values for a personal device from the only one controller node responsible for the personal device. The backend system and network may then be configured to determine the current position for the personal device based on the received Bluetooth broadcast signal strength values for the personal device and return the determined position to the only one controller node responsible for the personal device.

In embodiments, each of said plurality of personal devices is configured to transmit Bluetooth broadcast signals including alarm data in response to a trigger event determined by the respective personal device to have occurred, wherein the alarm data is adapted to indicate the occurrence of said trigger event to any node within short-range wireless communication range receiving the Bluetooth broadcast signal and is transmitted by the respective personal device while having an established Bluetooth connection with their respectively assigned only one connection node.

In embodiments, each of said plurality of personal devices is further configured to, in addition, transmit data including redundant alarm data in response to the determined occurrence of the trigger event via the established Bluetooth connection with their respectively assigned only one connection node. The respectively assigned only one connection node may then be configured to, in response to receiving the alarm data via said established Bluetooth connection, at least one of transmit and forward data including alarm data via said network, e.g. IP-based network, to the respectively assigned only one controller node, where the alarm data is adapted to indicate to the only one controller node that the trigger event has occurred.

In embodiments, the system comprises at least one mobile communication device and a backend system and network configured to receive alarm data indicating the occurred trigger event from the only one controller node responsible for the personal device which determined that the trigger event occurred. The backend system may then be configured to transmit alarm data to the at least one mobile communication device of an individual such as a staff member of the facility in response to receiving the alarm data from the only one controller node.

In embodiments, the backend system and network is further configured to transmit the alarm data to the at least one mobile communication device via a network separate from the network, e.g. IP-based network, used by the at least three network nodes to exchange data.

### BRIEF DESCRIPTION OF DRAWINGS

Preferred embodiments of a system according to the technology disclosed will be described more in detail below with reference to the accompanying drawings wherein:

FIG. 1 schematically shows an example of facilities where a system comprising a number of network nodes have been installed, in accordance with one or more embodiments described herein.

FIG. 2 is a schematic illustration of a system for monitoring an individual within predetermined facilities.

FIG. 3 is an example flow diagram of a method for monitoring an individual within predetermined facilities, in accordance with one or more embodiments described herein.

FIG. 4 schematically illustrates a method for monitoring an individual within predetermined facilities, in accordance with one or more embodiments described herein.

FIG. 5 schematically illustrates a method for monitoring an individual within predetermined facilities, in accordance with one or more embodiments described herein.

FIG. 6 schematically illustrates a method for monitoring an individual within predetermined facilities, in accordance with one or more embodiments described herein.

Embodiments of the present disclosure and their advantages are best understood by referring to the detailed description that follows. It should be appreciated that like reference numerals are used to identify like elements illustrated in one or more of the figures.

### DETAILED DESCRIPTION

In the drawings, similar details are denoted with the same reference number throughout the different embodiments. In the various embodiments of the system, e.g. monitoring system, according to the technology disclosed, the different subsystems are denoted. The “boxes”/subsystems shown in the drawings are by way of example only and can within the scope of the technology disclosed be arranged in any other way or combination.

The systems and ad-hoc networks known in the art, e.g. piconets or SON, does not disclose a node network where nodes share data or information which each other prior to the dynamic assignment of roles to the nodes.

The systems and ad-hoc networks known in the art, e.g. piconets or SON, does not disclose a node network where data (traffic) is separated into the communication between the nodes, which takes place using the IP address and via an IP-based network, e.g. via Wi-Fi, and the communication between the personal devices communicate and the nodes in the node network, which uses a short-range wireless communication protocol such as Bluetooth.

In embodiments, the technology disclosed provides a fast response yet robust solution for acting on an identified event by parallel and/or redundant data transmission of event data representing or reflecting the event via both an established short-range wireless connection with one of the nodes and via short-range wireless broadcast messages from the personal device to the nodes which are within short-range wireless communication range.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via Bluetooth broadcast transmissions from the personal device to the nodes which are within Bluetooth communication range.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via a Bluetooth broadcast message transmitted from the personal device to the nodes which are within Bluetooth communication range.

In embodiments, the technology disclosed provides a solution for dynamically and accurately determining the indoor position or location for a personal device without any use of high energy-consuming GPS in the personal devices or beacons, by measuring, by each of the network nodes which are within short-range communication range with the personal device, the signal strength of short-range wireless broadcast messages, e.g. Bluetooth broadcast messages, received from the personal device.

In embodiments, the controller node for the personal device may then receive, via the IP-based network, data and/or information representing or reflecting the measured signal strengths from each of the plurality of other nodes within short-range communication range with the personal device and, based on the data and/or information, determine the current position or location for the personal device.

In various embodiments, the controller node may forward the obtained data and/or information representing or reflecting the measured signal strengths from each of the plurality of other nodes, and/or other movement sensor data obtained, to a backend network which determines the current position or location for the personal device.

In various embodiments, the controller may in addition use other obtained sensor data, e.g. sensor data from the movement sensor of the personal device (e.g. an accelerometer), to determine and/or calculate the current position and/or location for the personal device.

In certain embodiments, the current position or location for the personal device may at least partly be determined by a trained machine learning algorithm used by the controller node or a backend system communicatively coupled to the controller node, since machine learning is an efficient way of evaluating complex signals in situations where large amounts of data is collected. The trained machine learning algorithm may then use at least the signal strength values received from the other nodes and/or movement sensor data detected by the personal device to determine the current position or location for the personal device.

In embodiments, the technology disclosed provides a roaming solution for dynamically changing the connection node for a personal device, by measuring, by each of the network nodes which are within short-range communication range with the personal device, the signal strength of short-range wireless broadcast messages, e.g. Bluetooth broadcast messages, received from the personal device.

In embodiments, the controller node for the personal device may then receive, via the IP-based network, data and/or information representing or reflecting the measured signal strengths from each of the plurality of other nodes within short-range communication range with the personal device and, based on the data and/or information, determine to change connection node for the personal device.

In various embodiments, the controller node may forward the obtained data and/or information representing or reflecting the measured signal strengths from each of the plurality of other nodes, and/or other movement sensor data obtained, to a backend network which determines that the connection node for the personal device should be changed.

In various embodiments, the controller node may in addition use other obtained sensor data, e.g. sensor data from a movement sensor of the personal device, e.g. an accelerometer, to determine that the connection node for the personal device should be changed.

In certain embodiments, the change of connection node for the personal device may at least partly be determined by a trained machine learning algorithm used by the controller node or a backend system communicatively coupled to the controller node, since machine learning is an efficient way of evaluating complex signals in situations where large amounts of data is collected. The trained machine learning algorithm may then use at least the signal strength values received from the other nodes and/or movement sensor data detected by the personal device to determine that the connection node for the personal device should be changed.

The systems and networks, e.g. piconets or SON, known in the art further does not disclose a system, e.g. monitoring system, comprising a node network and personal devices with broadcast capabilities which is self-organizing and scalable by dynamically assigning roles to the individual nodes, e.g. continuously and automatically assigning roles to the nodes, where each assignment of a role to a node relates to managing and control of only one of the personal devices. In particular, the systems and network solutions known in the art are silent to dynamically assigning and changing roles to the nodes on two levels, a control level where the node has the sole responsibility for a personal device and a connection level where the node is responsible for establishing a connection or link with the personal device.

The ad-hoc networks known in the art does not disclose or describe a node network where the nodes communicate internally via their IP addresses, but where the communication between personal devices and the network nodes takes place via a short-range wireless communication such as Bluetooth. This gives a much higher bandwidth capacity for the communication between the personal devices and the network nodes. This also enables the node network to be scalable and more distributed, since communication between network nodes can take place via the IP-based network connecting the nodes.

To address the identified problems with the prior art, the technology disclosed proposes a system, e.g. monitoring system, and node network for dynamically assigning roles to the nodes of the network on two levels, a control level and connect level.

The technology disclosed introduces a node network where the nodes communicate internally via their IP addresses, but where the communication between personal devices and nodes takes place via short-range wireless communication such as Bluetooth. This gives a better load balancing and much higher bandwidth capacity for the communication between the personal devices and the nodes. This also enables the node network to be more scalable and distributed, since communication between nodes can take place via an IP-based network.

The system, e.g. monitoring system, comprising the node network of the technology disclosed is highly scalable and provides for safe and fast responses to events, obtained data and the movements of the individuals, yet is robust in that each of the personal devices always has a reliable short-range wireless connection to the node network which is not disturbed or compete for short-range communication bandwidth with other devices connected to the same node of the node network.

In aspects, the technology disclosed allows for faster and more accurate decision-making and improved load balancing within a network by providing methods and a system comprising personal devices and a network of nodes for dynamically assigning one controller node and one connection node for each personal device. In embodiments, the controller node for a personal device is dynamically assigned or changed based on a common decision model known to the network nodes and which may contain input values or parameters. The input values or parameters may be changed with the data distributed and shared between the network nodes. In certain embodiments of the technology disclosed, the consensus model as such with its input values or parameters may also be dynamically adjusted and changed over time.

In embodiments, the technology disclosed relates to a system comprising a node network which is self-organization by including a dynamic assignment of a node as uploader node to the internet or backend system.

In certain embodiments, the technology disclosed relates to a system comprising a node network which is self-organization by assigning a “stand-by” node to take over from the assigned node, e.g. a stand-by node which is ready to take over from an assigned controller node, an assigned connection node and/or an assigned uploader node.

In certain embodiments, the assignment of a stand-by controller node may use the same common decision model as used for the assignment of the controller node where the decision model may, or may not, use input or parameter data values obtained from the personal devices and/or nodes of the network to determine which of the at least three nodes is going to be the stand-by node. The input or parameter data values may change the determining of which of the network nodes is going to be the stand-by controller node for the personal device.

In certain embodiments, the assignment, by the controller node, of a stand-by connection node to take over from the assigned connection node may be at least partly based on signal strengths of short-range wireless broadcast signals which are measured by a plurality of network nodes within short-range communication range with the personal device transmitting the short-range wireless broadcast signals. The assignment of stand-by connection node by the controller node may further use a load-balancing algorithm which may consider at least one of the number of personal devices each of the network nodes is controller node for, the number of short-range wireless connections in a certain location or geographical sub-area of the facility and the number of personal devices in a certain location or geographical sub-area of the facility.

The faster and more accurate decision-making is at least partly achieved by the method of assigning, for each personal device, one controller node with sole responsibility for the personal device, where the controller node is provided with all data or information associated with the personal device the controller node is responsible for and which is received, obtained and/or measured by the network nodes.

The faster and more accurate decision-making may be further achieved by the controller node making all decisions for the personal device, e.g. the decision to set an alert or transmit alarm data or the assignment of connection node with which the personal device is establishing a short-range wireless communication link such as a Bluetooth connection. The connection node may then have an established connection with the personal device as long as the controller node considers it appropriate, e.g. as long as the signal strength measured by the connection node is above a certain threshold value and/or a signal strength measured by the connection node is higher than signal strengths measured by the other nodes of the network.

Improved load-balancing is achieved by having the controller node with sole responsibility for a personal device dynamically assigning and changing connection node for the personal device at least partly based on load-balancing factors such as the current number of established short-range wireless connections for a node, e.g. as compared to the number of connections for other nodes, to thereby distribute the amount of short-range wireless data transmitted locally.

In certain aspects, the system and self-organizing network of the technology disclosed separating the data traffic in the IP-based network traffic from the short-range wireless communication performed by the personal devices provides an improved solution over ad-hoc networks and many state of the art self-organizing networks in that the transmission of data between the nodes used for configuring and self-organizing the network nodes, e.g. data used for assigning different roles to the nodes, is performed via the IP-based network and not via the short-range wireless protocol the personal device are using for providing the network nodes with data, thereby does not load, affect or interfere with the short-range wireless radio traffic, e.g. the Bluetooth radio traffic.

Benefits of the technology disclosed include that the system, self-organizing network and methods provide each of the personal devices with improved short-range wireless connections by continuously and dynamically changing which of the network nodes is the sole connection node having the only short-range wireless connection, e.g. Bluetooth connection, to a personal device at least partly based on measured signal strengths for the currently established short-range wireless connection and/or short-range signal strengths measured by the individual nodes of the network.

Further benefits of the technology disclosed include that the controller node is provided with all data associated with the personal device it is responsible for in that the all data or information associated with the personal device, or data or information representing or reflecting the obtained data or information, is forwarded from the other network nodes to the controller node, including the data received and measured by the currently assigned connection node for the personal device.

In embodiments, the technology disclosed provides a fast response yet robust solution for acting on an identified event by parallel redundant data transmission of event data representing or reflecting the event via both an established short-range wireless connection with one of the nodes and via short-range wireless broadcast messages from the personal device to the nodes which are within short-range wireless communication range.

Bluetooth is a short-range wireless technology standard that allows data to be exchanged between fixed and mobile devices. The technology uses short wavelength radio waves from 2.4 to 2.485 GHz. Unlike other forms of connectivity such as wi-fi or 4G, Bluetooth carries connections between devices and other devices as opposed to carrying data to and from the internet.

BLE stands for Bluetooth Low Energy and is a form of wireless communication designed especially for short-range communication. BLE is very similar to Wi-Fi in the sense that it allows devices to communicate with each other. However, BLE is meant for situations where battery life is preferred over high data transfer speeds. Wi-Fi uses multiple parts of the IEEE 802 protocol family and is designed to interwork seamlessly with its wired sibling Ethernet. Compatible devices can network through wireless access points to each other as well as to wired devices and the Internet. The different versions of Wi-Fi are specified by various IEEE 802.11 protocol standards, with the different radio technologies determining radio bands, and the maximum ranges, and speeds that may be achieved. Wi-Fi most commonly uses the 2.4 GHz (120 mm) UHF and 5 GHz (60 mm) SHF ISM radio bands; these bands are subdivided into multiple channels. Channels can be shared between networks but only one transmitter can locally transmit on a channel at any moment in time.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via Bluetooth broadcast transmissions from the personal device to the nodes which are within Bluetooth communication range.

In embodiments, the technology disclosed provides a fast response yet robust solution for acting on an identified event, e.g. the event of pressing an alarm button on a personal device such as a wristband, by parallel redundant data transmission of event data (e.g. alarm data) representing or reflecting the event via both an established Bluetooth connection with one of the nodes and via a Bluetooth broadcast message transmitted from the personal device to the nodes which are within Bluetooth communication range.

In embodiments, the technology disclosed provides a solution for dynamically and accurately determining the indoor position or location, e.g. current room, for a personal device without any use of high energy-consuming GPS in the personal devices or iBeacon technology, by measuring, by each of the network nodes which are within short-range communication range with the personal device, the signal strength of short-range wireless broadcast messages, e.g. Bluetooth broadcast messages, received from the personal device.

In embodiments, the controller node for the personal device may then receive, via the IP-based network, data and/or information representing or reflecting the measured signal strengths from each of the plurality of other nodes within short-range communication range with the personal device and, based on the data and/or information, determine the current position or location for the personal device.

In various embodiments, the controller node may forward the obtained data and/or information representing or reflecting the measured signal strengths from each of the plurality of other nodes, and/or other movement sensor data obtained, to a backend network which determines the current position or location for the personal device.

In various embodiments, the controller may in addition use other obtained sensor data, e.g. sensor data from the movement sensor of the personal device (e.g. an accelerometer), to determine and/or calculate the current position and/or location for the personal device.

The controller node, or a local or remote processing arrangement receiving the measured signals strengths from the controller node (and optionally also sensor data obtained by a sensor of the personal device), may then be configured to determine the current position or location for the personal device by comparing the signal strengths of the broadcast signals measured by the other nodes (and the controller node) and then determine the position or location based on the comparison. In embodiments, the position or location of the node measuring the highest signal strength for the broadcast signal is determined to be the position or location for the personal device, or the position or location for the personal device may be determined or calculated using signals strengths measured by a plurality of nodes, e.g. by using triangulation, or the position or location for the personal device may be determined or calculated using at least one signals strength of a broadcast signal and sensor data obtained by a sensor of the personal device, e.g. a motion sensor or a pressure sensor.

In certain embodiments, the current position or location for the personal device may at least partly be determined by a trained machine learning algorithm used by the controller node or a backend system communicatively coupled to the controller node, since machine learning is an efficient way of evaluating obtained sensor data and event data, e.g. in situations where large amounts of data is collected. The trained machine learning algorithm may then use at least the signal strength values received from the other nodes and/or movement sensor data detected by the personal device to determine the current position or location for the personal device.

In embodiments, the technology disclosed provides a roaming solution for dynamically and continuously changing the connection node for a personal device, by measuring, by each of the network nodes which are within short-range communication range with the personal device, the signal strength of short-range wireless broadcast messages, e.g. Bluetooth broadcast messages, received from the personal device.

In certain embodiments, the setting of an alarm or the change of health state and/or alert state for the personal device may at least partly be determined by a trained machine learning algorithm used by the controller node or a backend system communicatively coupled to the controller node, since machine learning is an efficient way of evaluating sensor data in situations where large amounts of data is collected. The trained machine learning algorithm may then use at least the signal strength values received from the nodes (including the current controller node and the current connection node for the personal device if any of these nodes are within short-range wireless communication range with the personal device to receive the broadcasts) and/or movement sensor data detected by the personal device to determine that the connection node for the personal device should be changed.

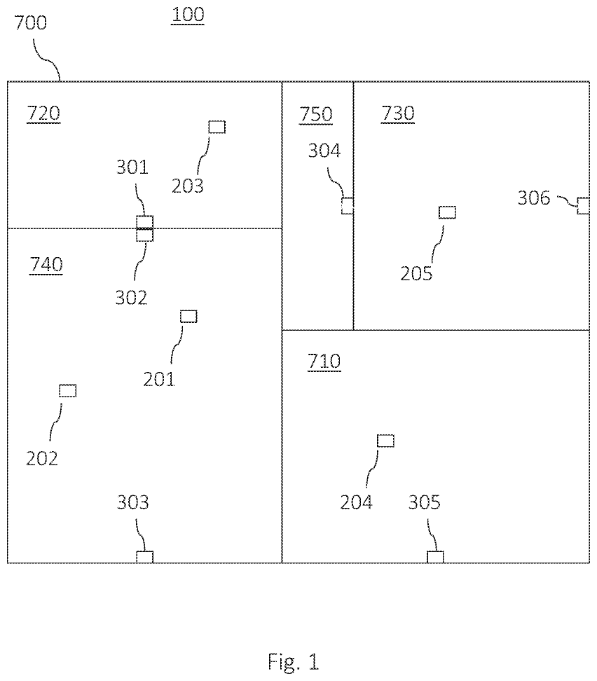

FIG. 1 schematically shows an example of a facility 700 where a system 100 comprising a number of network nodes (301, 302, 303, 304, 305, 306) has been installed, in accordance with one or more embodiments described herein. The illustrated system 100 comprises a plurality of personal devices (201, 202, 203, 204, 205) with short-range wireless communication capability. Such facility 700 may e.g. be an apartment where a monitored individual is residing. Such an apartment 700 may e.g. comprise a kitchen 710, a bathroom 720, a bedroom 730, a sitting room 740, and a hallway 750. The system 100 may in this case be used by relatives, home care companies or the like.

The facility 700 may also be other types of quarters where a monitored individual may be residing, such as e.g. a nursing home, where there may be a private space for the monitored individual, including e.g. a bedroom 730, as well as public spaces that are shared by a plurality of monitored individuals.

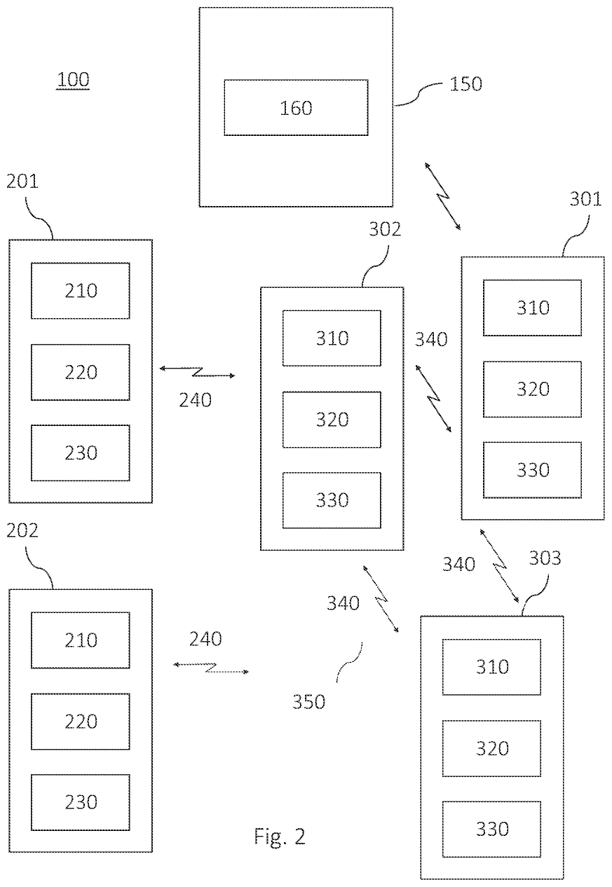

FIG. 2 is a schematic illustration of a system 100 for monitoring an individual within predetermined facilities 700, in accordance with one or more embodiments described herein. The illustrated system 100 comprises a plurality of personal devices (201, 202), a plurality of network nodes (301, 302, 303), and a processing arrangement 150, e.g. a locally-installed processing arrangement or a remote processing arrangement such as a backend system. The personal devices (201, 202) may be a wristband or other portable device that is carried or worn by an individual, e.g. a necklace or a clip worn by a patient in a nursing home. The illustrated personal devices (201, 202) comprise a processing device 210, at least one sensor and/or user input means 220, and a personal device communication interface 230. The at least one sensor may, for example, include a motion sensor, a temperature sensor, a health sensor such as a body temperature sensor, a pressure sensor and/or any other type of sensor and the user input means may comprise a button or voice input means that the individual can press or activate to set off an alarm and/or transmit event data indicating the occurrence of a certain event. The illustrated network nodes (301, 302, 303) comprise a node processing device 310, a storage means 320, and a node communication interface 330. The illustrated processing arrangement 150 comprises a processing device 160, which may e.g. be comprised in a web server. However, the system 100 does not necessarily comprise a processing arrangement 150.

Information regarding the layout of the facilities 700, such as the plan of the different rooms and the location of the node modules 300, is preferably stored in the storage means 320. The storage means 320 does not have to be comprised in the network node—it can be comprised in another part of the system such as a locally-installed processing arrangement or a remote processing arrangement such as a backend system, or be a separate device or module.

According to embodiments of the technology disclosed, the at least one processing device 310 of the network nodes (301, 302, 303) may be arranged to: receive status data, position data or signal strength values from another node of the network, or sensor data, input data or event data originating from the at least one sensor and/or user input means 220 in the personal device 200; determine whether the received data indicates a need for certain action associated with the personal device, e.g. changing health state for the individual wearing the personal device and/or alert state among a plurality of predetermined alert states. As an example, the alert states may comprise at least a FALL alert state, to be used if a probable fall has been detected for the monitored individual, and an OUT OF BED alert state, to be used if it has been detected that the monitored individual is probably getting out of bed.

In various embodiments, the at least one processing device 210, 310, 160 processing the sensor data, input data or event data may be the personal device processing device 210, the node processing device 310, or another processing device, such as a local or remote processing device 160. The at least one processing device may also be a combination of any number of processing devices, so that some of the processing takes place in one processing device and some of the processing takes place in one or more other processing devices. It is thus not necessary for all of the processing to take place in the same processing device.

If weighing factors are used, these weighing factors may be determined by a machine learning system, since this is an efficient way of evaluating complex signals in situations where large amounts of data is collected.

One way of making such a determination is to determine a probability that an alert should be set based on the received event data and/or sensor data, and a probability that the determined location fulfils the predetermined location condition, and then combine these probabilities using weighing factors, which may e.g. be determined by a machine learning system.

The same applies to other combinations, such as e.g. the determination of the position and/or location for the individual wearing the personal device by involving also signals from other sensors of the personal device such as a movement sensor 220, and/or the setting of alerts based also on activity states. These determinations may also be done by determining probabilities and using weighing factors to combine them, which may e.g. be determined by a machine learning system.

In embodiments, the determining of a position and/or location for the individual wearing the personal device by the controller node (301) may e.g. be based on the received signal strength indication (RSSI) of short-range wireless broadcast signals transmitted by the personal device and which are received by the network nodes within short-range communication range with the personal device. The network nodes within short-range communication range then measure the RSSI of short-range wireless broadcast signals and forwards the RSSI to the controller node for the personal device. If the RRSI of short-range wireless broadcast signals received by more than one network node is determined, the location of the personal device may be more reliably determined based on e.g. triangulation. Machine learning may also be used for this determination. The measured RSSI values of short-range wireless broadcast signals transmitted by a personal device may be used by the controller node assigned to a personal device for determining the current position and/or location for the personal device in connection with transmitting position or location data together with alert state data or when setting an alert or alarm for the individual wearing the personal device. Thus, the decision by the controller node for a particular personal device to set an alarm for the individual or to transmit alert state data or alarm data may be based on broadcast signal strength values measured and received from other nodes within short-range communication range with the personal device (including the current controller node and the current connection node for the personal device if any of these nodes are within short-range wireless communication range with the personal device to receive the broadcasts).

The personal devices 200 may also comprise other sensors 220, such as e.g. a pressure sensor, temperature sensor or medical sensor. It is known to use a pressure sensor in a personal device to detect a fall based on a change in pressure. However, a pressure sensor may also be involved in the determination of the location of the individual, e.g. when location data is transmitted together with health state data and/or alert state data to a local or remote processing arrangement, e.g. a backend system, configured to set an alert or an alarm. If the facilities 700 comprise several floors, and the node modules 300 are arranged in the ceilings or high up on the walls, the RSSI may give erroneous results due to the signals travelling between the floors. In this situation, a pressure sensor may be used to determine the floor on which the individual is located.

In order to improve the determination of the need for e.g. changing health state for the individual and/or setting an alert even further, the determination of the location may be improved by involving also signals from a movement sensor 220. The movement sensor 220 may e.g. be used as a pedometer, so that the signals from the movement sensor 220 indicates how many steps the individual has taken. If the individual is determined to be in a certain location (such as e.g. the bathroom 720), and the amount of steps required for moving to this location from a previously determined location (such as e.g. the bedroom 730) is more than the amount of steps that have been detected by the movement sensor 220, it is likely that the determined location is incorrect. The at least one processing device 210, 310, 160 may thus be arranged to determine the location of the personal device 200 based also on an indication of the movement of the individual in relation to a previously determined location, based on sensor data from the at least one motion sensor 220.

The processing of the sensor data from the at least one sensor 220 may be done by a trained machine learning algorithm used by the controller node or a local or remote processing arrangement communicatively connected to the personal device. When many individuals are monitored using many personal devices 200, large amounts of sensor data is collected from the sensors 220 in these personal devices 200. Very efficient evaluation of this sensor data may be done using machine learning, in order to determine a suitable machine learning algorithm.

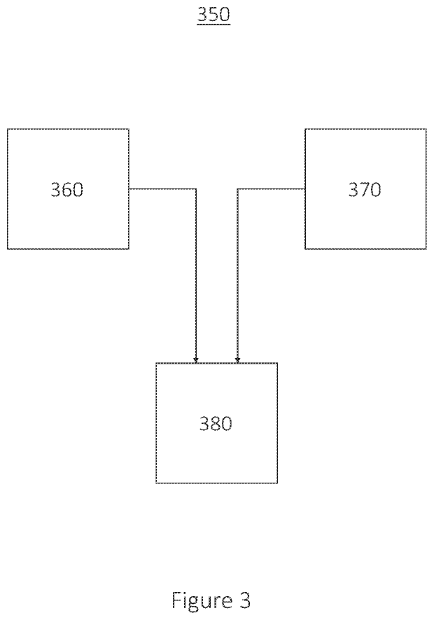

FIG. 3 is an example flow diagram of a method for monitoring an individual within predetermined facilities in order to determine whether there is a need for responding to status data, sensor data and/or event data received by the controller node, e.g. changing health state for the individual and/or setting an alert, in accordance with one or more embodiments described herein. The flow 350 may e.g. be as follows:

Step 360: Determine, by the controller node for a personal device 200, routing and/or load-balancing aspects or factors associated with at least two of the at least three network nodes in the network, where the routing and/or load-balancing aspects may consider at least one of the number of personal devices each of the network nodes is controller node for, the number of short-range wireless connections in a certain location or geographical sub-area of the facility and the number of personal devices in a certain location or geographical sub-area of a facility.

Step 370: Receive, by the controller node for a certain personal device, signal strength values for short-range wireless signals transmitted by a personal device 200 and which are measured and forwarded to the controller node by a plurality of network nodes 300.

Step 380: Combine these inputs into a resulting determination, by the controller node, of whether to take actions in response to the received signal strength values, e.g. to change the health state and/or alert state for the individual wearing the personal device, set an alert or alarm for the personal device and/or individual wearing the personal device, or transmit alert or alarm data associated with the personal device and/or individual wearing the personal device to a local or remote processing arrangement

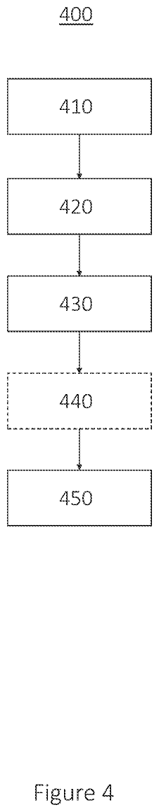

FIG. 4 schematically illustrates a method 400 for monitoring an individual within predetermined facilities 700 in order to determine the current position and/or location for the personal device. The method 400 may include the following steps:

Step 410: transmitting short-range wireless broadcast signals from a personal device 200.

Step 420: measuring, by a plurality of network nodes 300, signal strength values of the short-range wireless broadcast signals transmitted by the personal device 200 and which are received by the plurality of network nodes 300.

Step 430: forwarding the measured signal strength values measured by the respective of the plurality of network nodes 300 to the controller node for the personal device.

Step 450: determining the current position and/or location of the personal device 200 within the facilities 700 based at least on the received signal strength values.

In certain embodiments, the method (400) may comprise:

Step 440: retrieving information from a storage means 320 regarding the layout of the facilities 700 within which the monitoring of the individual takes place.

In these certain embodiments, the step 450 of determining the location of the personal device 200 within the facilities 700 is based at least on the received signal strength values together with the information retrieved from a storage means 320 regarding the layout of the facilities 700 within which the monitoring of the individual takes place.

In the embodiments, the step 450 of determining the current position and/or location for the personal device may be based at least partly on received sensor data, e.g. obtained by a pressure sensor or motion sensor of the personal device, indicates a specific location and/or a specific node to be the new connection node and/or whether the determined location fulfils a predetermined location condition, indicating whether the individual is located in a certain room, such as e.g. the bedroom 630.

The combination of motion information with location information improves the determination of changing connection node for the personal device.

The determining of whether the received sensor data indicates a specific location among a plurality of predetermined location may e.g. be done by a trained machine learning algorithm of the controller node or a locally-installed processing arrangement or a remote processing arrangement such as a backend system communicatively connected to the controller node, since machine learning is an efficient way of evaluating complex signals in situations where large amounts of data is collected and moving patterns of the individuals wearing the personal devices are repetitive and/or predictable.

In embodiments, the distance between the personal device 200 and the at least one network node 300 may e.g. be determined based on the received or obtained signal strength indication (RSSI) of the short-range wireless signal transmitted by the personal device 200 and received by the plurality of network nodes, since the received signal strength will be lower if the distance increases. If the RRSI for more than one network node is determined, the location may be more exactly determined based on e.g. triangulation. Machine learning may also be used for this determination.

In embodiments, the determining 450 of the current position and/or location of the personal device 200 is based also on an indication of the movement of the individual in relation to a previously determined location, based on sensor data from the at least one sensor 220. If the individual is determined to be in a certain location, and the movement required for moving to this location from a previously determined location is more than the movement that has been detected by the at least one movement sensor, it is likely that the determined location is incorrect.

In embodiments, the at least one sensor 220 is an accelerometer. However, other types of motion sensors may also be used.

In embodiments, the personal device 200 is a wristworn device, such as e.g. a wristband. The personal device 200 may however be any type of device that may be worn by an individual, such as e.g. a necklace or a clip.

In embodiments, communication between the personal device 200 and the at least one node 300 takes place using a personal device communication interface 230 and a node communication interface 330. In embodiments, the technology disclosed may include continuously determining and updating an activity state for the monitored individual by continuously receiving sensor data from the personal device, e.g. health sensor data obtained by a medical sensor of the personal device.

The activity state may e.g. categorize the current activity of the individual. Activity states may e.g. be LYING IN BED, LYING ON COUCH, LYING ON FLOOR, SITTING, STANDING, WALKING, RUNNING, RIDING A WHEELCHAIR, WALKING UP/DOWN STAIRS, IN THE BATHROOM. There may also be a specific activity state for when the individual is not wearing the personal device, since no other activity state can then be set. There may be a specific sensor, such as e.g. a contact sensor, a temperature sensor or a heart rate sensor, for determining whether the individual is wearing the personal device. Such a sensor may of course also be used for other purposes.

In embodiments, the technology disclosed may include continuously determining and updating a health state for the monitored individual by continuously receiving sensor data from the personal device worn by the individual, e.g. health sensor data obtained by at least one medical sensor of the personal device.

The determining of the activity state and/or the health state of the individual wearing the personal device may e.g. be done by a trained machine learning algorithm, since machine learning is an efficient way of evaluating complex signals in situations where large amounts of data is collected.

In embodiments, the personal device 200 may also comprise sensors, such as e.g. a motion sensor or pressure sensor. It is known to use a pressure sensor in a personal device to detect a fall based on a change in pressure. However, a pressure sensor may also be involved in the determination, by the controller, to change connection node for the personal device 200 carried by the individual. If the facilities 700 comprise several floors, and the network nodes 300 are arranged in the ceilings or high up on the walls, the RSSI may give erroneous results due to the signals travelling between the floors. In this situation, data obtained by a pressure sensor of the personal device received by the controller node may be used by the controller node to determine the floor on which the individual is located which, in turn, is an input factor to the step of determining, by the controller node for the personal device 200, whether to change connection node for the personal device 200.

In embodiments and in order to improve the determination of whether to change connection node for a personal device 200, the determination of the location and/or whether to change connection node may be improved by involving also signals from a sensor 220 of the personal device 200. The sensor 220 may e.g. be a movement sensor used as a pedometer, so that the signals from the movement sensor 220 indicates how many steps the individual has taken. If the individual is determined to be in a certain location (such as e.g. the bathroom 720), and the amount of steps required for moving to this location from a previously determined location (such as e.g. the bedroom 730) is more than the amount of steps that have been detected by the movement sensor 220, it is likely that the determined location is incorrect. The at least one processing device 310 of a network node (301, 302, 303, 304, 305, 306) may thus be arranged to determine the location of the personal device 200 influencing the decision by the controller node, or processing arrangement, whether to change alert state and/or health state for the personal device 200 based also on an indication of the movement of the individual in relation to a previously determined location, based on sensor data from the at least one sensor 220.

The processing of the sensor data from the at least one sensor 220 may be done by a trained machine learning algorithm. When many individuals are monitored using many personal devices 200, large amounts of sensor data is collected from the sensors 220 in these personal devices 200. Very efficient evaluation of this sensor data may be done using machine learning, in order to determine a suitable machine learning algorithm.

The updating of the activity state and/or health state for the monitored individual may be incremental, so that only differences in relation to the previously determined activity state and/or health state need to be determined.

The determination of the location for the personal device 200 and/or the by the controller node to change connection node for the personal device 200 may e.g. be done by a trained machine learning algorithm. Machine learning is an efficient way of evaluating complex signals in situations where large amounts of data is collected.

When the determining of whether to change alert state and/or health state is based on received sensor data indicates a certain location and whether the determined location fulfils a predetermined location condition, the predetermined location condition may be an absolute condition, such that if the determined location does not fulfil the predetermined location condition, the controller node will, or will not, determine to change alert state and/or health state for the personal device 200. However, there may instead be weighing factors, so that if the received sensor data strongly indicates a specific location, and/or the determined location does not strongly indicate a specific location (that does not fulfil the predetermined location condition), the controller node will, or will not, determine to change alert state and/or health state for the personal device 200.

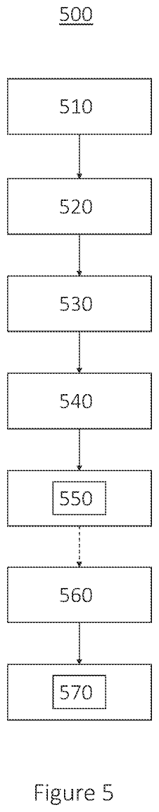

FIG. 5 schematically illustrates embodiments of the technology disclosed including a method (500) for monitoring an individual wearing a personal device within predetermined facilities (700), the method (500) comprising:
a. establishing (510), by a connection node assigned to the personal device, a short-range wireless communication link with the personal device;
b. transmitting (520), from the personal device and via said established short-range wireless communication link, a message to the connection node assigned to the personal device;
c. receiving (530), by the connection node, the message, wherein the message contains data including at least one of status data, sensor data and event data associated with the personal device;
d. transmitting (540), from the assigned connection node to the controller node for the personal device, data including the at least one of status data, sensor data and event data associated with the personal device;
e. transmitting (550), from the personal device, at least one short-range wireless broadcast signal, wherein the at least one short-range wireless broadcast signal is transmitted while the short-range wireless communication link between the connection node and the personal device is active;
f. forwarding (560), from the network nodes within short-range communication range with the personal device and which receive the at least one short-range wireless broadcast signal to the controller node, sensor data and/or event data contained in the at least one short-range wireless broadcast signal; and
g. determining (570), by the controller node assigned to the personal device, a position, a location, a health state and/or an alert state based on at least one of the at least one of status data, sensor data and event data received from the assigned connection node and the sensor data and/or event data forwarded by the network nodes within short-range communication range with the personal device.

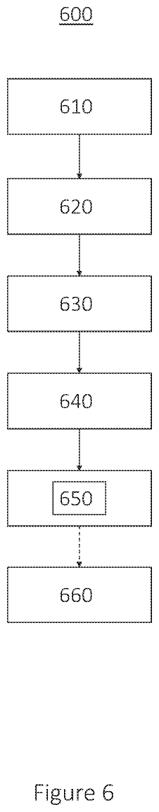

FIG. 6 schematically illustrates embodiments of the technology disclosed including a method (600) for monitoring an individual within predetermined facilities (700), the method (600) comprising:
a. transmitting (610), by the personal device, at least one short-range wireless broadcast signal;
b. receiving (620), by a plurality of nodes within short-range wireless communication range with the personal device, at least one of the at least one short-range wireless broadcast signals;
c. measuring (630), by each of the plurality of nodes, signal strengths of the received at least one short-range wireless broadcast signal;
d. transmitting (640), from each of the plurality of nodes and via the IP-based network connecting the at least three nodes of the node network, data to the controller node, wherein the data indicates the measured signal strengths values;
e. receiving (650), by the controller node assigned to the personal device and via the IP-based network, the transmitted data indicating the measured signal strengths values; and
f. determining (660), by the controller node, to transmit data to a processing arrangement and/or set an alert state or health state for the personal device and/or the individual wearing the personal device at least partly based on the received data indicating the measured broadcast signal strengths values.

In aspects, the system is configured so that the new connection node may be a different node from the current controller node for the personal device but may also be the controller node for the personal device.

In embodiments of the technology disclosed, the system is configured so that the connection node assigned to a personal device is never the assigned controller node for the personal device.

In aspects, the technology disclosed relates to a method and system for monitoring an individual within predetermined facilities (700):
receiving, by the sole connection node assigned to the personal device, a message comprising sensor data obtained by at least one sensor (220) arranged in the personal device (200);
forwarding, by the connection node and to the controller node assigned to the personal device, a message comprising the sensor data or data reflecting the sensor data;
determining, by the controller node whether the received sensor data indicates certain event data, a specific health state and/or a specific alert state among a plurality of predetermined alert states, where the alert states may comprising at least a FALL alert state, to be used if a probable fall has been detected for the monitored individual, and an OUT OF BED alert state, to be used if it has been detected that the monitored individual is probably getting out of bed;
receiving, by a plurality of nodes within short-range wireless communication range with the personal device, short-range wireless broadcast signals from the personal device;
measuring, by each of the plurality of nodes, signal strengths values of the received short-range wireless broadcast signals;
forwarding, by each of the plurality of nodes and to the controller node assigned to the personal device, a message containing the signal strengths values of the received short-range wireless broadcast signals;
determining, by the controller node, the location of the personal device (200) within the facilities (700) based at least on the received signal strengths values of the short-range wireless broadcast signals together with information retrieved from a storage means (320) regarding the layout of the facilities (700) within which the monitoring of the individual takes place; and
setting an alert and/or change a health state based at least on whether the received sensor data indicates a specific event, health state and/or an alert state and whether the determined location fulfils a predetermined location condition, indicating whether the individual is located in a certain room, such as e.g. the bedroom (630).

According to embodiments of the various methods mentioned above, the determining of the location of the personal device (200) is based also on an indication of the movement of the individual in relation to a previously determined location, based on sensor data from the at least one sensor (220), e.g. a motion sensor and/or a pressure sensor.

According to embodiments of the technology disclosed and mentioned above, the method further comprising continuously determining and updating an activity state and/or a heath state for the monitored individual.

According to embodiments of the technology disclosed and mentioned above, the method further the change of health state and/or setting of the alert is based also on at least one previously determined health state or activity state. In the example of setting an alert state, the setting of an OUT OF BED alert state may also depend on whether the previous activity state was a LYING IN BED alert state, or so that the setting of a FALL alert state also depends on that the previous activity state was not any type of LYING activity state.

According to embodiments of the technology disclosed and mentioned above, the changed health state and/or setting of the alert is based also on the time period for which the individual has had a specific activity state, such as e.g. for how long the individual has been in the activity state STANDING.

According to embodiments of the technology disclosed and mentioned above, determining of the changed health state and/or activity state is done by a trained machine learning algorithm.

According to embodiments of the technology disclosed and mentioned above, the method further comprises sending an alarm signal if a predetermined alert or health state is set.

In embodiments, the communication between the personal device (200) and the at least one network node (300) takes place using a personal device communication interface (230) and a network node communication interface (330).

In aspects, the technology disclosed relates to a system (100) for monitoring an individual within predetermined facilities (700), the system (100) comprising:
a personal device (200) to be worn by the monitored individual, the personal device (200) comprising at least one sensor or input means (220);
at least one network node (300), with which the personal device (200) is arranged to communicate via an established short-range wireless connection;
a storage means (320); and
at least one processing device (210, 310, 160), arranged to:
   receive sensor data, event data and/or input data from the at least one sensor and/or input means (220) in the personal device (200);
   determine whether the received sensor data, event data and/or input data indicates a specific health state and/or an alert state among a plurality of health states and/or predetermined alert states; and;
   set a health state and/or an alert based at least on whether the received sensor data, event data and/or input data indicates a health state and/or an alert state.

In embodiments, the system (100) and controller node for the personal device (200) are configured to set a health state and/or an alert based at least on whether a determined location fulfils a predetermined location condition, indicating whether the individual is located in a certain room, such as e.g. the bedroom (630).

In embodiments, the system (100) and controller node for the personal device (200) are configured to determine the distance between the personal device (200) and the at least one network node (300), e.g. based on signal strength values measured by at least one of the network nodes.

In embodiments, the system (100) and controller node for the personal device (200) are configured to determine the location of the personal device (200) within the facilities (700) based at least on the determined distance together with information retrieved from the storage means (320) regarding the layout of the facilities (700) within which the monitoring of the individual takes place.

In embodiments, the system (100) and the at least one processing device (210, 310, 160) are arranged to continuously determine and update a health state and/or an activity state for the monitored individual based on received sensor data is indicating a specific event, health state and/or an alert state.

In embodiments, the personal device (200) comprises at least one motion sensor (220). In certain embodiment the at least one motion sensor (220) is an accelerometer.

In embodiments, the personal device (200) comprises at least one medical sensor (220), or health sensor, where the medical sensor (220) is adapted to obtain health sensor data to be used for determining the current health status or health state of the individual wearing the personal device.

In embodiments, the personal device (200) is a wristworn device, such as e.g. a wristband, clip or necklace.

An accelerometer, is a device that measures proper acceleration. Proper acceleration, being the acceleration (or rate of change of velocity) of a body in its own instantaneous rest frame, is not the same as coordinate acceleration, being the acceleration in a fixed coordinate system.

The input from the movement sensor based on sensor data indicating an acceleration beyond a certain threshold value and/or a certain changed movement pattern for the personal device may trigger the short-range wireless transmission of event data from the personal device.

1: A method in a system including a network comprising at least two network nodes and a plurality of personal devices with short-range wireless communication capabilities, said method comprising:
a. assigning one node among the at least two network nodes to be controller node for a personal device;
b. assigning a connection node for establishing a short-range wireless communication link between said connection node and said personal device;
c. establishing, by said assigned connection node, a short-range wireless communication link with said personal device;
d. transmitting, from said personal device and via said established short-range wireless communication link, a message to said connection node;
e. receiving, by said connection node, said message, wherein said message contains data including at least one of status data, sensor data and event data associated with said personal device;
f. transmitting, from the assigned connection node to the controller node for said personal device, data including said at least one of status data, sensor data and event data associated with said personal device; and
g. transmitting, from said personal device, at least one short-range wireless broadcast signal, wherein said at least one short-range wireless broadcast signal is transmitted while said short-range wireless communication link between said connection node and said personal device is active.

2: The method of claim 1, wherein said at least one of status data, sensor data and event data associated with said personal device is provided to said controller node by forwarding said received message from said connection node or by transmitting, from said connection node, a message including data corresponding to, or at least partly based on, said received at least one of status data, sensor data and event data associated with said personal device.

3: The method of claim 1, wherein said message is forwarded or transmitted via an IP-based network connecting the at least two network nodes.

4: The method of claim 3, wherein said IP-based network is a separate network and uses a different communication protocol from the communication protocol used for said established short-range wireless communication link between said connection node and said personal device.

5: The method of claim 1, wherein said established short-range wireless communication link between said connection node and said personal device is a bidirectional short-range wireless communication link.

6: The method of claim 1, wherein said message from said connection node to said controller node and said at least one short-range wireless broadcast signal transmitted from said personal device are containing data indicating the same sensor data and/or event data.

7: The method of claim 1, wherein said message from said connection node to said controller node is containing data indicating at least one of the activation of an input means of the personal device, data indicating a trigger event and sensor data indicating a health state and/or alert state for the personal device and/or the individual wearing the personal device.

8: The method of claim 1, wherein said at least one short-range wireless broadcast signal transmitted from said personal device is containing data indicating at least one of the activation of an input means of the personal device, data indicating a trigger event and sensor data indicating a health state and/or alert state for the personal device and/or the individual wearing the personal device.

9: The method of claim 1, wherein said message from said connection node is containing sensor data and/or event data, said method further comprising:
a. determining, by said controller node and at least partly based on said received sensor data and event data associated with said personal device, a specific alert state and/or a health state for the individual wearing the personal device.

10: The method of claim 1, wherein said message from said connection node is containing sensor data and/or event data, said method further comprising:
a. transmitting, from said controller node to a local or remote processing arrangement, data indicating a changed health state and/or alert state for the individual wearing the personal device.

11: The method of claim 1, wherein said message from said connection node is containing sensor data and/or event data, said method further comprising:
a. transmitting, from said controller node, said sensor data and/or event data associated with said to a local or remote processing arrangement; and
b. determining, by said local or remote processing arrangement, the current health state and/or alert state for the individual wearing the personal device based on said sensor data and/or event data, wherein processing of the sensor data for determining the current health state and/or alert state is performed by said local or remote processing arrangement using a trained machine learning algorithm.

12: The method of claim 9, further comprising:
a. determining, by said local or remote processing arrangement, a changed health state and/or alert state for the individual wearing the personal device controller node and at least partly based on said received sensor data and event data associated with said personal device is indicating, a specific alert state and/or a health state for the individual wearing the personal device; and
b. transmitting, from said controller node to a local or remote processing arrangement, data indicating a changed health state and/or alert state for the individual wearing the personal device.

13: The method of claim 10, further comprising:
a. setting, by said local or remote processing arrangement, an alarm for the personal device and/or individual wearing the personal device, wherein said setting of an alarm is at least partly based on the determining of a changed health state and/or alert state for the individual wearing the personal device.

14: The method of claim 1, further comprising:
a. receiving, by at least one network node within short-range wireless communication range with said personal device, said at least one short-range wireless broadcast signal;
b. measuring, by each of said at least one network node within short-range wireless communication range with said personal device, the signal strengths of said at least one short-range wireless broadcast signal;
c. transmitting, from each of said at least one network node within short-range wireless communication range with said personal device and to said controller node, at least one message containing data indicating the respective signal strengths of the at least one short-range wireless broadcast signal measured by the respective at least one network node within short-range wireless communication range with said personal device; and
receiving, by said controller node for said personal device, at least one message containing data indicating the respective signal strengths, wherein a message is received from the respective of the at least one network node and is indicating the respective signal strengths measured by the respective at least one node within short-range wireless communication range with said personal device.

15: The method of claim 14, said method further comprising:
a. determining, by said controller node and at least partly based on said data indicating said measured signal strengths, the current position and/or location for the personal device and/or the individual wearing the personal device.

16: The method of claim 14, further comprising:
a. transmitting, from said controller node to a local or remote processing arrangement, data indicating said measured signal strengths for the respective at least one node; and
b. determining, by said local or remote processing arrangement, the current position and/or location for said personal device and/or the individual wearing the personal device, wherein said determining is at least partly based on said received data indicating said measured signal strengths for the respective at least one node.

17: The method of claim 15, further comprising:
a. obtaining, by at least one of a motion sensor and a pressure sensor of said personal device, sensor data indicating a pressure and/or a movement associated with the personal device and/or the individual wearing the personal device; and
b. transmitting, from said personal device via said established data indicating said sensor data obtained by said personal device is received and forwarded by said connection node to said controller node, and wherein said determining of said current position and/or location is at least partly based on sensor data obtained by a motion sensor and/or a pressure sensor of said personal device, wherein said data indicating said sensor data is received and forwarded by said connection node to said controller node.

18: The method of claim 1, wherein said message from said connection node is containing sensor data and/or event data, said method further comprising:
a. transmitting, from said controller node, said sensor data and/or event data associated with said to a local or remote processing arrangement; and
b. determining, by said local or remote processing arrangement, the current health state and/or alert state for the individual wearing the personal device based on said sensor data and/or event data, wherein processing of the sensor data for determining the current health state and/or alert state is performed by said local or remote processing arrangement using a trained machine learning algorithm.

19: The method of claim 16, further comprising:
a. determining, by said local or remote processing arrangement, a changed health state and/or alert state for the individual wearing the personal device controller node and at least partly based on said received sensor data and event data associated with said personal device is indicating, a specific alert state and/or a health state for the individual wearing the personal device; and
b. transmitting, from said controller node to a local or remote processing arrangement, data indicating a changed health state and/or alert state for the individual wearing the personal device.

20: A system including a network comprising at least two network nodes and a plurality of personal devices with short-range wireless communication capabilities, said system is configured to assign a connection node among said at least two network nodes to each of said plurality of personal devices, wherein the connection node is responsible establishing a short-range wireless communication link with their respective personal device, and wherein each of said plurality of personal devices is configured to transmit short-range wireless broadcast signals while their respective established short-range wireless connection with their respectively assigned connection node is active.

21: The system according to claim 20, wherein said system is configured to assign a controller node for the respective of said plurality of personal device, and wherein each controller node for the respective personal device is configured to assign a connection node for the personal device and to receive, from the other network nodes, status data, sensor data and/or event data associated with the personal device it is responsible for.

22: The system according to claim 20, wherein said at least two network nodes are configured to share data with the other nodes via an IP-based network connecting the at least two network nodes.

23: The system according to claim 22, wherein said IP-based network is a separate network and is configured to use a different communication protocol from the communication protocol used for said established short-range wireless communication link between said connection node and said personal device.

24: The system according to claim 20, wherein said established short-range wireless communication link between said connection node and said personal device is a bidirectional short-range wireless communication link.

25: The system according to claim 20, wherein each of said plurality of personal devices is configured to transmit data indicating the same obtained sensor data and/or event data both via their respective established short-range wireless connection with their respectively assigned connection node and via short-range wireless broadcast signals, wherein said same sensor data and/or event data is data indicating at least one of the activation of an input means of the personal device, data indicating a trigger event and sensor data indicating a health state and/or alert state for the personal device and/or the individual wearing the personal device.

26: The system according to claim 21, wherein said controller node for the respective personal device is configured to determine a specific alert state and/or a health state for the individual wearing the personal device at least partly based on received sensor data and/or event data associated with the personal device.

27: The system according to claim 20, wherein each of said at least two network nodes is configured to first measure signal strengths of short-range wireless broadcast signals received from a personal device and then automatically transmit or forward data indicating said measured signal strengths to the identified controller node responsible for the personal device transmitting the short-range wireless broadcast signals.

28: The system according to claim 27, wherein the controller node for the respective personal device is configured to determine a position and/or a location for their respective personal device at least partly based on the received data indicating said measured signal strengths.
